(19) United States
(12) Patent Application Publication
(10) Pub. No.: US 2024/0052097 A1
(43) Pub. Date: Feb. 15, 2024

(54) SYNTHESIS OF PEG-BASED THIOL-NORBORNENE HYDROGELS WITH TUNABLE HYDROYLITIC DEGRADATION PROPERTIES

(71) Applicant: The Trustees of Indiana University, Bloomington, IN (US)

(72) Inventors: Chien-Chi Lin, Bloomington, IN (US); Fang-Yi Lin, Bloomington, IN (US)

(21) Appl. No.: 18/264,508

(22) PCT Filed: Feb. 4, 2022
(86) PCT No.: PCT/US2022/015174

§ 371 Date: Aug. 7, 2023

Related U.S. Application Data
(60) Provisional application No. 63/147,152, filed on Feb. 8, 2021.

(51) Int. Cl. C08G 65/332; C08G 65/333; A61K 47/34; C08G 65/334; C08J 3/075; A61L 27/18; A61L 27/52; A61L 27/56; A61L 27/38; C12N 5/0775

(52) U.S. Cl. CPC C08G 65/3324; C08G 65/3331; A61K 47/34; C08G 65/334; C08J 3/075; A61L 27/18; A61L 27/52; A61L 27/56; A61L 27/3834; C12N 5/0662; C08G 2210/00; C08J 2371/02; C12N 2533/30

(57) ABSTRACT

Methods for synthesizing a hydrogel having tunable hydrolylic degradation kinetics according to certain embodiments include synthesizing a norborene-functionalized polyethylene glycol macromer having an ester linkage between a polyethylene glycol domain of the macromer and a norbornene domain of the macromer and having a carboxyl group on the norbornene; and reacting a quantity of such macromers with a quantity of dithiol molecules by an ultraviolet-initiated photo-gelation reaction to yield a hydrogel. Such macromers and such hydrogels form additional aspects of the disclosure, as do a wide variety of methods for tuning and for using such hydrogels.

### CROSS-REFERENCE TO RELATED APPLICATIONS

This application claims priority to U.S. Provisional Application No. 63/147,152, which was filed Feb. 8, 2021, the entire content of which is incorporated by reference herein.

### STATEMENT ON FEDERALLY SPONSORED RESEARCH OR DEVELOPMENT

This invention was made with government support under CA227737 awarded by National Institutes of Health and under 1452390 awarded by the National Science Foundation. The Government has certain rights in the invention.

### FIELD

Embodiments disclosed herein are directed to hydrogels, and more particularly to hydrogels with tunable hydrolytic degradation properties.

### BACKGROUND

Polyethylene glycol (PEG)-based hydrogels are widely used in tissue engineering and biofabrication for their excellent biocompatibility, adaptable cross-linking mechanisms, and readily tunable physiochemical properties. Degradable PEG-based hydrogels have played a pivotal role in biomaterials and tissue engineering applications and considerable efforts have been dedicated to designing PEG-based hydrogels with tunable degradation. These PEG-based hydrogels are particularly desirable in tissue regeneration, controlled release, and biosensor applications.

Conjugation of functional groups (e.g., acrylate, norbornene, vinylsolfone, maleimide) on the termini of PEG chains is necessary to permit their cross-linking into hydrogels. On the other hand, labile motifs (e.g., polyletic acid, PLA) are routinely copolymerized with functionalized PEG to render the otherwise stable network hydrolytically labile. Another approach to increase hydrolytie degradation properties of PEG-based hydrogels that has been explored was to synthesize ester-containing cross-finkers with different hydrolytic susceptibility, such as groups with steric hindrance, local hydrophobicity, and electron-withdrawing moieties.

One category of PEG-based hydrogel that has gained a great deal of attention includes hydrogels fabricated by orthogonal thiol-norbornene photo-polymerization, one example of which is made using multiarm PEG-norbornene (“PEGNB”) macromers. PEGNB macromers can be cross-linked into a network using a multifunctional thiol cross-linker through a radical-mediated thiol-norbornene reaction initiated by ultraviolet light, visible light, or enzymatic reaction.

To make PEGNB macromers that are more hydrolytically labile, which is important for many desired uses for such hydrogels, reactions that form ester linkages between the PEG component of the macromer and the norbornene component of the macromer are desired. Conventional processes for synthesis of PEONB with ester linkages (referred to herein as “PEGeNB”), however, are extremely time-consuming and laborious, and require the use and handling of noxious chemicals, such as 5-norbornene-2-carboxylic acid (“NB-acid”), which is very unpleasant due to its pungent and extremely unpleasant odor. As a result of the unpleasant odor of NB-acid, conventional processes for synthesizing PEGeNB require stringent containment efforts during and after the synthesis process. Thus, while PEGeNB macromers are commercially available, they are expensive and, hence, are not a sustainable option, which significantly limits the widespread usage of the PEGeNB macromer.

Therefore, a need remains for development of improved processes for making PEGeNB macromers and for development of macromers and hydrogels having improved properties. The present disclosure addresses these needs.

### SUMMARY

The present disclosure provides methods for synthesizing a hydrogel baying tunable hydrolytic degradation kinetics. According to certain embodiments, a method includes synthesizing a norbornene-functionalized polyethylene glycol macromer having an ester linkage between a polyethylene glycol domain of the macromer and a norbornene domain of the macromer and either (i) having a carboxyl group on the norbornene domain or (ii) wherein at least one norbornene domain of the macromer is connected through a carboxamide linkage to a functional group. According to some embodiments, a method includes reacting a quantity of a norbornene-functionalized polyethylene glycol macromer with a quantity of dithiol molecules by an ultraviolet-initiated photo-gelation reaction to yield a hydrogel, wherein the norbornene-functionalized polyethylene glycol macromer comprises an ester linkage between a polyethylene glycol domain of the macromer and either (i) having a carboxyl group on the norbornene domain or (ii) wherein at least one norbornene domain of the macromer is connected through a carboxamide linkage to a functional group.

According to an embodiment, a method for synthesizing a norbornene-functionalized polyethylene glycol macromer includes: (i) providing a polyethylene glycol reactant comprising at least one polyethylene glycol domain with a terminal hydroxyl group: and (ii) reacting the polyethylene glycol reactant with carbic anhydride in the presence of a nucleophilic catalyst to yield a norbornene-functionalized polyethylene glycol macromer having an ester linkage between a polyethylene glycol domain of the macromer and a norbornene domain of the macromer. wherein the norbomnene domain comprises a carboxyl group.

Some embodiments include any other method disclosed herein wherein the polyethylene glycol reactant comprises a multi-arm polyethylene glycol molecule. Some embodiments include any other method disclosed herein wherein the polyethylene glycol reactant comprises a polyethylene glycol molecule selected from the group consisting of a 2-arm polyethylene glycol molecole, a 3-arm polyethylene glycol molecule, a 4-arm polyethylene glycol molecule, a 6-arm polyethylene glycol molecule, an 8-arm polyethylene glycol molecule and combinations thereof. Some embodiments include any other method disclosed herein wherein the nucleophilic catalyst comprises 4-dimethylaminopyridine. Some embodiments include any other method disclosed herein wherein the norbornene-functionalized polyethylene glycol macromer comprises a structure as shown in Formula 1, wherein n=from about 10 to about 300, and R₁ comprises a member selected from the group consisting of a second arm of PEG, optionally additional arms of PEG, a macromolecule, a PEGylated inorganic compound that includes a tail of PEG, and combinations thereof.

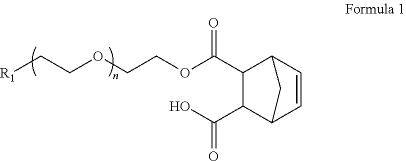

Some embodiments include any other method disclosed herein, and further comprising conjugating the carboxyl group of the norbornene domain with an amine-containing molecule to provide a modified macromer. Some embodiments include any other method disclosed berein wherein the amine-containing molecule comprises a member selected from the group consisting of L-3,4-dihydroxyphenethylamine, tyramine, isopropylamine and combinations thereof. Some embodiments include any other method disclosed herein wherein the modified macromer comprises at least one component having a structure as shown in Formula 2 wherein n=from about 10 to about 300, X is an aromatic group or an aliphatic group, and R₁ comprises a member selected from the group consisting of a second arm of PEG, optionally additional arms of PEG, a macromolecule, a PEOylated inorganic compound that includes a tail of PEG, and combinations thereof.

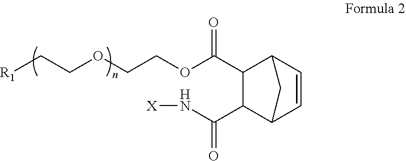

Some embodiments include any other method disclosed herein wherein X is selected from the group consisting of a functional group of Formula 3, a functional group of Formula 4, a functional group of Formula 5, a functional group of Formula 6, and a functional group of Formula 7.

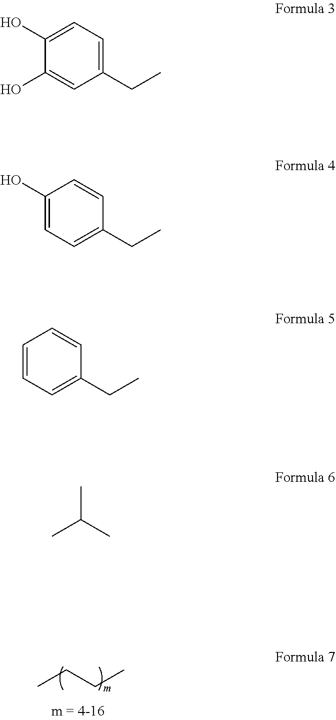

The present disclosure also provides a method for modifying a norbornene-functionalized polyethylene glycol macromer that includes: (i) providing a norbornene-functionalized polyethylene glycol macromer having an ester linkage between a polyethylene glycol domain of the macromer and a norbornene domain of the macromer, wherein the norbornene domain comprises a carboxyl group; and (ii) conjugating the carboxyl group of the norbornene domain with an amine-containing molecule to provide a modified macromer.

The present disclosure also provides a macromer for producing a hydrogel. the macromer comprising a multi-arm polyethylene glycol core having at least two polyethylene glycol domains, each polyethylene glycol domain connected through an ester linkage to a norbornene moiety, wherein at least one norbornene moiety of the macromer comprises a carboxyl group. Some embodiments include any other macromer disclosed herein wherein each of the at least two polyethylene glycol domains comprises from about 10 to about 300 —CH2—CH2—O— units. Some embodiments include any other macromer disclosed herein wherein the multi-arm polyethylene glycol core has a number of polyethylene glycol domains selected from the group consisting of two, three, four and eight. Some embodiments include any other macromer disclosed herein wherein the macromer comprises at least one component having a structure as shown in Formula 1 wherein n=from about 10 to about 300, and R₁ comprises a member selected from the group consisting of a second arm of PEO, optionally additional arms of PEO, a macromolecule, a PEGylated inorganic compound that includes a tail of PEG, and combinations thereof.

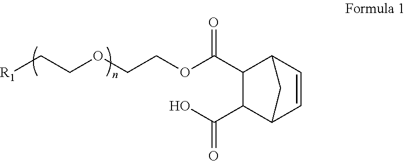

Some embodiments include any other macromer disclosed herein wherein at least two norbornene moieties of the macromer comprise a carboxyl group.

The present disclosure also provides a macromer for producing a hydrogel, the macromer comprising a multi-arm polyethylene glycol core having at least two polyethylene glycol domains, each polyethylene glycol domain connected through an ester linkage to a norbornene moiety, wherein at least one norbornene moiety of the macromer is connected through a carboxamide linkage to a functional group. Some embodiments include any other macromer disclosed herein wherein the macromer comprises at least one component having a structure as shown in Formula 2 wherein n=from about 10 to about 300, X is an aromatic group or an aliphatic group, and R₁ comprises a member selected from the group consisting of a second arm of PEG, optionally additional arms of PEG, a macromolecule, a PEGylated inorganic compound that includes a tail of PEG, and combinations thereof.

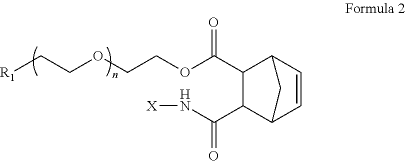

Some embodiments include any other macromer disclosed herein wherein X is selected from the group consisting of a functional group of Formula 3, a functional group of Formula 4, a functional group of Formula 5, a functional group of Formula 6, and a functional group of Formula 7.

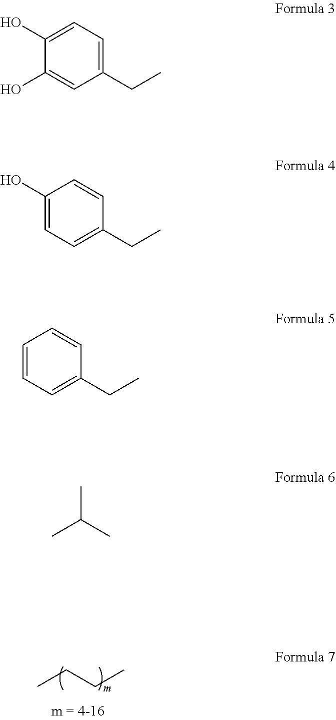

Some embodiments include any other macromer disclosed herein wherein at least two norbornene moieties of the macromer are connected through a carboxamide linkage to a functional group.

The present disclosure also provides a method for making a hydrogel that includes: (i) providing a first quantity of macromers comprising a norbornene-functionalized polyethylene glycol macromer as disclosed herein and combinations thereof; (ii) providing a second quantity of dithiol molecules; (iii) mixing the first quantity of macromers with the second quantity of dithiol molecules to provide a reaction mixture; and (iv) applying ultraviolet radiation to the reaction mixture to initiate a photo-gelation reaction to yield a hydrogel. Some embodiments include any other method disclosed herein wherein the dithiol molecule is operable as a hydrogel cross-linker. Some embodiments include any other method disclosed herein wherein the dithiof molecule is selected from the group consisting of 1,4-dithiothreitol, 4-arm thiolated PEG, a peptide that includes more than one cysteine residue, a natural or thiolated protein such as thiolated gelatin, thiolated hyaluronic acid, and thiolated collagens, and combinations thereof. Some embodiments include any other method disclosed herein wherein the dithiol molecule comprises dithiothreitol. Some embodiments include any other method disclosed herein wherein the hydrogel comprises an orthogonal thiol-norbornene hydrogel. Some embodiments include any other method disclosed herein wherein the hydrogel has a hydrolyte degradation rate of from about 0.0045 to about 1.46 h⁻¹. Some embodiments include any other method disclosed herein wherein the hydrolytic degradation rate is independent of pH.

The present disclosure also provides a hydrogel produced according to any method disclosed herein. The present disclosure also provides a hydrogel comprising a cross-linked network composed of: (i) a plurality of multiarm polyethylene glycol structures, each of said plurality of multiarm polyethylene glycol structures including at least two polyethyle glycol domains covalently bonded to one another directly or through a covalent connecting structure comprising one or more atoms; (ii) a plurality of norbornene domains; and (iii) a plurality of dithio-derived cross-linkers. Some embodiments include any other method disclosed herein wherein each of said plurality of polyethylene glycol domains is covalently bonded to one of said plurality of a norbornene domains via an ester linkage. Some embodiments include any other method disclosed herein wherein each of said plurality of norbornene domains includes a carboxyl group or a carboxamide linking a functional group to said norbornene domain. Some embodiments include any other method disclosed berein wherein each of said dithio-derived cross-linkers is covalenty bonded to two of said plurality of norbornene domains. Some embodiments include any other method disclosed herein wherein said hydrogel exhibits faster hydrolytic degradation compared to a hydrogel having the same structure except lacking the carboxyl group or carboxamide.

The present disclosure also includes uses of any hydrogel disclosed herein for a purpose selected from the group consisting of as a substrate for short-term 2D cell culturing, 3D in situ encapsulation of viable cells, controlled delivery of drugs and other bioactive materials, tissue engineering, tissue regeneration, creating micropores in a bulk polyethylene glycol-based hydrogel, creating a sacrificial material in a 3D bioprinted article, creating a sacrificial material in a photolithography-based biofabrication product, and creating a sacrificial bioink for extrusion-based or photolithography-based 3D bioproducts.

The present disclosure also includes a method for tuning hydrogel degradation kinetics that includes: (i) mixing at least two norbornene-functionalized polyethylene macromers, at least one of the norbornene-functionalized polyethylene macromers selected from any macromer as disclosed herein; (ii) controlling the proportions of the at least two norbornene-functionalized polyethylene macromers based on at least one desired hydrogel characteristic selected from the group consisting of cross-link density, hydrolytie degradation kinetics, shear modulus and swelling ratio; (iii) mixing the at least two norbornene-functionalized polyethylene macromers with a predetermined quantity of dithiol molecules to provide a reaction mixture; and (iv) applying ultraviolet radiation to the reaction mixture to initiate a photo-gelation reaction to yield a hydrogel having tuned degradation kinetics.

These and other embodiments, forms, features, and aspects of the disclosure will become more apparent through reference to the following description, the accompanying figures, and the claims. This summary is not intended to identify key or essential features of the claimed subject matter, nor is it intended to be used as an aid in limiting the scope of the claimed subject matter. Additionally, it is to be understood that the features of the various embodiments described berein are not mutually exclusive and can exist in various combinations and permutations. Furthermore, it is envisioned that alternative embodiments may combine features of two or more of the above-summarized embodiments. Further embodiments, forms, features, and aspects of the present application shall become apparent from the description and figures provided herewith.

### BRIEF DESCRIPTION OF THE DRAWINGS

The concepts described herein are illustrative by way of example and not by way of limitation in the accompanying figures. For simplicity and clarity of illustration, elements illustrated in the figures are not necessarily drawn to scale. Where considered appropriate, references labels have been repeated among the figures to indicate corresponding or analogous elements.

FIG. (“FIG.”) 1 shows synthesis and characterization data for PEGNB_{CA} hydrogels, in accordance with embodiments disclosed herein.

FIG. 2 shows ¹H NMR spectra (Panel A) and quantification of residual Cys in PEGeNB-CA, in accordance with embodiments disclosed herein.

FIG. 3 depicts hydrolytic degradation of PEGNM and PEGNB_{CA} hydrogels, in accordance with embodiments disclosed herein.

FIG. 4 depicts the degradation of PEGNB and PEGNB_{CA} hydrogels at 37° ° C. by swelling ratio, in accordance with embodiments disclosed herein.

FIG. S depicts the live/dead staining and confocal imaging of cell-laden PEGNB_{CA} peptide hydrogels at Day 1 and at Day 7, in accordance with embodiments disclosed herein.

FIG. 6 shows the cross-linking and degradation of PEGNB, PEGNB_{CA}, and PEGNB-X hydrogels, in accordance with embodiments disclosed herein.

FIG. 7 provides ¹H NMR spectra of PEGNB_{CA}, PEGNB-D, PEGNB-T, and PEGNB-1, in accordance with embodiments disclosed herein.

FIG. 8 provides the degradation of PEGNB-X hydrogels at room temperature, in accordance with embodiments disclosed herein.

FIG. 9 demonstrates the control of degradation of PEGNB hydrogels via cross-linking with various cross-linkers and mixing in PEGNB-D, in accordance with embodiments disclosed herein.

FIG. 10 provides the initial shear moduli and the degradation of 10 wt % PEGNBCA/PEGNB-D-DTT hydrogels with varying PEONB-D content, in accordance with embodiments disclosed herein.

FIG. 11 shows a 10 wt % PEGNB-D 25% hydrogel on Day 0 and Day 7, in accordance with embodiments disclosed herein.

FIG. 12 provides live/dead staining and confocal imaging of cell-laden 25%/75% PEGNB-D/PEGNBca-peptide hydrogels at Day 0. Day 1, and Day 2, in accordance with embodiments disclosed berein.

FIG. 13 provides live/dead staining and confocal imaging of cell-laden PEGNB-D/PEGNB_{CA}-peptide hydrogels at a 50%, 75%, and 100% PEGNB-D ratio in the composites, in accordance with embodiments disclosed herein.

FIG. 14 demonstrates the tunable crosslinking and rapid degradation of PEGNB-Dopa hydrogels, in accordance with embodiments disclosed herein.

FIG. 15 shows the fabrication and characterization of macroporous hydrogels, in accordance with embodiments disclosed berein.

FIG. 16 provides characterization data obtained in a control experiment using non-degradable PEGNB microgels.

FIG. 17 shows moduli changes and the swelling ratio of macroporous hydrogels, in accordance with embodiments disclosed herein.

FIG. 18 shows A549 spheroid formation within macroporous hydrogels, in accordance with embodiments disclosed herein.

FIG. 19 provides characterization data of AS49 cells and COLO-357 cells, in accordance with embodiments disclosed herein.

FIG. 20 provides characterization data for mMSC solid spheroid formation within macroporous hydrogels, in accordance with embodiments disclosed herein.

FIG. 21 shows the effect of bulk hydrogel properties on the formation of multicellular mMSC structures, in accordance with embodiments disclosed herein.

FIG. 22 provides characterization data for MSCs formed in accordance with embodiments disclosed herein.

FIG. 23 provides fluorescence imaging of solid spheroids and hollow spheres formed with MSCs, in accordance with embodiments disclosed herein.

FIG. 24 provides characterization data using the AKT pathway, in accordance with embodiments disclosed herein.

FIG. 25 provides an antibody array for detecting arrays of growth factors and inflammatory cytokines, in accordance with embodiments disclosed herein.

FIG. 26 provides heatmaps for growth factors and inflammation cytokines secreted in the CM produced from hMSCs encapsulated as single cells or assembled as spheroids in the macroporous hydrogels, in accordance with embodiments disclosed herein.

### DETAILED DESCRIPTION

Although the concepts of the present disclosure are susceptible to various modifications and alternative forms, specific embodiments have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended clairos.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should be further appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described. Additionally, it should be appreciated that items included in a list in the form of “at least one of A. B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary.

In the drawings, some structural or method features may be shown in specific arrangements and/or orderings. However, it should be appreciated that such specific arrangements and/or orderings may not he required. Rather, in some embodiments, such features may be arranged in a different manner and/or order than shown in the illustrative figures unless indicated to the contrary. Additionally, the inclusion of a structural or method feature in a particular figure is not meant to imply that such feature is required in all embodiments and, in some embodiments, may not be included or may be combined with other features.

Unless defined otherwise, all technical and scientific terms used herein have the same meaning as is commonly understood by one of ordinary skill in the art to which this disclosure belongs. All patents, applicationa, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

The singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The terms “including,” “containing,” and “comprising” are used in their open, non-limiting sense.

To provide a more concise description, some of the quantitative expressions given berein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. In some embodiments, the term “about” in reference to a number or range of numbers is understood to mean the stated number and numbers +/−10% thereof, or 10% below the lower listed limit and 10% above the higher listed limit for the values listed for a range.

Certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed berein.

In general, this disclosure involves the synthesis of a series of PEGeNB macromers. As described further herein, examples of such macromers include poly(ethylene glycol)-ester-norbornene-carboxylic acid (referred to herein as “PEGeNB-CA” and/or “PEGNB_{CA} ”) and its derivatives (referred to herein as “PEGeNB-X” and/or “PEGNBₓ” (examples of which include Dopamide, Tyramide, Isopropylamide, etc. linked to the PEGeNB macromer at X)). In certain aspects of this disclosure, the PEGeNB-CA and PEGeNB-X macromers are used to fabricate cytocompatible hydrogels with highly tunable hydrolytic degradation kineties. Compared to existing PEG-based hydrogels with similar compositions and physical characteristics, and formed with identical cross-linking methods, the PEGeNB-CA and PEGeNB-X hydrogels can be cross-linked to similar elastic moduli but the PEGeNB-CA and PEGeNB-X hydrogels degrade hydrolytically at a much faster rate. In the work relating to the present disclosure, gels made using PEGeNB-CA and PEGeNB-X macromers were cross-linked to moderate and soft tissue-like stiffness (e.g., 0.15-10 kPa for 2.5-10 wt % PEGeNB-CA in water).

In addition to the discovery that hydrogels made with PEGeNB-CA and PEGeNB-X macromers exhibit rapid hydrolytie degradation kinetics, another aspect of this disclosure relates to the discovery that the PEGeNB-CA and PEGeNB-X macromers can be synthesized in a much shorter time than PEGeNB macromers previously synthesized without laborious macromer synthesis and modification, and while avoiding other problems associated with conventional synthesis techniques, such as the need to handle and contain noxious NB-acid. The discovery that PEGeNB-CA and PEGeNB-X hydrogels degraded rapidly without the inclusion of additional degradable macromer represents a significant improvement in the design and use of PEG-based hydrogels in tissue engineering and biofabrication applications.

In one aspect, this disclosure provides a synthesis method for a PEGeNB macromer that is significantly more efficient than, and that avoids the use of noxious chemicals that are used in, previously known processes. In one embodiment, a method for synthesizing a PEGeNB macromer includes (i) providing a polyethylene glycol reactant comprising at least one polyethylene glycol domain with a terminal hydroxyl group; and (ii) reacting the polyethylene glycol reactant with carbie anhydride in the presence of a nucleophilic catalyst to yield a PEGeNB macromer having an ester linkage between a polyethylene glycol domain of the macromer and a norbornene domain of the macromer, and wherein the norbornene domain comprises a carboxyl group. Carbic anhydride is an odorless, norbornene-derivative containing anhydride, which reacts with a hydroxyl-terminated polyethylene glycol domain through esterification. Compared with methods that use 5-norbornene-2-carboxylic acid, the methods disclosed herein are effective to produce similar PEGeNB macromers but with reduced synthesis time (approximately 2 days) as no PEG distillation and norbornene acid activation steps are required. In one embodiment, the polyethylene glycol domain comprises from about 10 to about 300 —CH₂—CH₂—O— units. With this method, significant quantities of PEGeNB can be produced within a time period of approximately two (2) days, compared to the approximately 5-day conventional processes. Moreover, the method avoids the use of noxious chemicals that are required in conventional processes. The methods of this disclosure therefore remove major obstacles to the commercial-scale synthesis of PEGeNB. and provide other advantages and benefits that will be described more fully herein.

In some embodiments, the polyethylene glycol reactant in the method comprises a multi-arm polyethylene glycol molecule. In some embodimenta, the polyethylene glycol reactant comprises a polyethylene glycol molecule selected from the group consisting of a 2-arm polyethylene glycol molecule, a 3-arm polyethylene glycol molecule, a 4-arm polyethylene glycol molecule, a 6-arm polyethylene glycol molecule and an 8-arm polyethylene glycol molecule. In some embodiments, the polyethylene glycol reactant comprises one or more other linear and branched polymers containing pendent hydroxyl groups, such as, for example and without limitation, vinyl alcohol or glycerol derivatives. As will be appreciated by a person of ordinary skill in the art, each polyethylene glycol arm of a polyethylene glycol molecule that includes a terminal hydroxyl group may be norbornene functionalized in the reaction. In some embodiments, each polyethylene glycol arm of the multi-arm polyethylene glycol molecule in the method includes a terminal hydroxyl group; however, this disclosure also contemplates multi-arm polyethylene glycol molecules in which some, but not all, of the polyethylene glycol arms include terminal hydroxyl groups.

In some embodiments, the nucleophilic catalyst comprises a pyridine-based catalyst or a tertiary amine-based catalyst. In some embodiments, the nucleophilic catalyst comprises a member selected from the group consisting of pyridine, p-pyrrolidinopyridine, 4-dimethylaminopyridine, trimethylamine, tricthylamine, diisopropylethylamine. In some embodiments, the nucleophilic catalyst comprises 4-dimethylaminopyridine.

Another unique feature of the methods described herein lies in the fact that the PEGeNB macromer produced by the reaction includes a norbornene domain that includes a carboxyl group. In some embodiments, the PEGeNB macromer comprises a structure as shown in Formula 1:

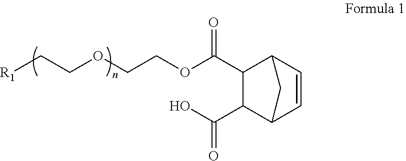

wherein n=from about 10 to about 300, and R₁ comprises a member selected from the group consisting of a second arm of PEG, optionally additional arms of PEG, a macromolecule, a PEGylated inorganic compound that includes a tail of PEG, and combinations thereof. As will be appreciated by a person of ordinary skill in the art, in an embodiment in which the polyethylene glycol reactant comprises a multi-arm polyethylene glycol molecule R₁ will include all other polyethylene glycol arms of the molecule and optionally may also include additional motifs, moieties or components. For example and without limitation, in a method embodiment that includes 4-arm polyethylene glycol molecule, R₁ includes three additional polyethylene glycol arms (and optionally additional motifs, moieties or components), in a method embodiment that includes 6-arm polyethylene glycol molecule, R₁ includes five additional polyethylene glycol arms (and optionally additional motifs, moieties or components) and in a method embodiment that includes an 8-arm polyethylene glycol molecule, R₁ includes seven additional polyethylene glycol arms (and optionally additional motifs, moieties or components). The present disclosure also contemplates, however, that R; may include no additional polyethylene glycol arms, and may include a wide variety of other motifs, moieties and components in alternative embodiments of the method. In some representative embodiments, R₁ may comprise block copolymers such as polyethylene glycol-b-propylene glycol-b-ethylene glycol) (pluronic), natural materials grafted with PEG such as PEG-g-chitosan and PEG-g-lipids. PEGylated inorganics such as PEG grafted gold, silver, copper nanoparticles, and PEO grafted metal organic frameworks.

One advantage of having a carboxyl group on the norbornene domain of a PEGeNB macromer synthesized in accordance with this disclosure is that the resulting macromer, referred to herein as a PEGeNB-CA macromer, has unexpected and advantageous properties. For example, when used to produce a hydrogel as described herein, a PEGeNB-CA macromer produces a hydrogel that is significantly more labile to hydrolytic degradation, i.e., that degrades much more quickly upon exposure to water than other PEGNB macromers previously described. As will be appreciated by a person of ordinary skill in the art, a higher the concentration of PEGeNB-CA macromers used in the reaction mixture produces a hydrogel having higher hydrolytie degradation kinetics than does a hydrogel produced from a reaction mixture having a lower concentration of PEGeNB-CA macromers.

The discovery that PEGeNB-CA macromers degrade much faster than conventional hydrogels made using PEGeNB macromers prepared in accordance with prior methods, without the inclusion of additional degradable macromer. represents a significant improvement in the utility of the PEGeNB-CA macromers for design and use of PBG-based hydrogels in tissue engineering applications. For comparison, it has been previousty shown that fully cross-linked PEGeNB-DTT hydrogels made using PEGeNB macromers made in accordance with prior methods degraded slowly via ester hydrolysis with a time frame of weeks to months. Interestingly, thiol-norbornene hydrogels cross-linked using PEGeNB-CA macromers as describe herein degraded (at 37° C.) completely in 8 days. In sum, PEGeNB-CA hydrogels degrade approximately 10-fold faster than the PEGeNB hydrogels previously described.

Another advantage of having a carboxyl group on the norbornene domain of a PEGeNB-CA macromer synthesized in accordance with this disclosure is that the PEGeNB-CA macromers can be further functionalized by reacting another compound with the carboxyl group. Thus, another aspect of the present disclosure comprises further functionalizing a PEGeNB-CA macromer by reacting (or conjugating) another compound with a carboxyl group on a norbornene domain of the macromer. In addition, a significant discovery disclosed berein is that the moiety selected for conjugation to the norbornene domain of the maeromer has a significant effect on the hydrolytic degradation kinetics of a hydrogel made using the macromer. For example, as discussed further herein, a macromer made by conjugating the carboxyl group with dopamine can be used to make a hydrogel that hydrolytically degrades up to 100 times faster than a hydrogel made with a PEGeNB-CA macromer.

In some embodiments, a PEGeNB-CA macromer as described herein, which includes a carboxyl group on the norbornene domain of the macromer, is reacted with an amine-containing molecule to provide a modified macromer (also referred to herein as a derivative macromer. a conjugated macromer. a further functionalized macromer, or PEGeNB-X). The reaction results in conjugating the carboxyl group of the norbornene domain with an amine-containing molecule to provide the modified macromer. In some embodiments, the amine-containing molecule comprises a primary amine or a secondary amine. In some embodiments, the amine-containing molecule comprises an aromatic amine or an aliphatic amine. In some embodiments, the amine-containing molecule comprises a member selected from the group consisting of L-3,4-dihydroxyphenethylamine, tyramine, isopropylamine, benzyl amine, stearamine, bexylamine and combinations thereof. In some embodiments, the amine-containing molecule comprises a member selected from the group consisting of L3,4-dihydroxyphenethylamine, tyramine and combinations thereof.

In some embodiments, the modified macromer comprises at least one component having a structure as shown in Formula 2:

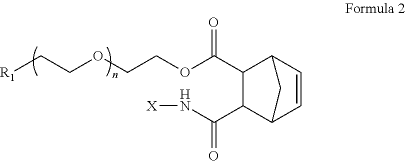

wherein n=from about 10 to about 300, X is group derived from reaction of the amine-containing molecule with the carboxyl group on the norbornene domain of a PEGeNB-CA macromer, and R₁ is as described above in connection with Formula 1. In some embodiments, X comprises an aromatic group or an aliphatic group. In some embodiments, X is selected from the group consisting of Formula 3, Formula 4, Formula 5, Formula 6, and Formula 7:

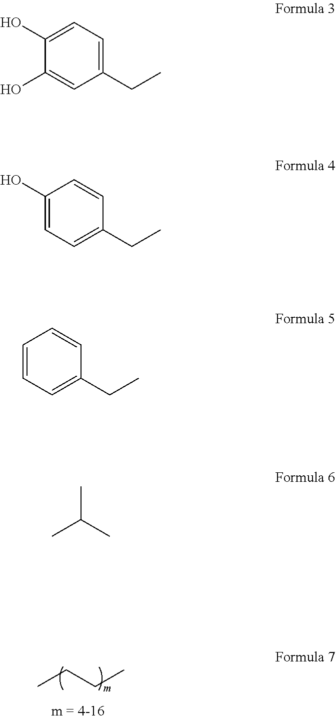

As indicated above, a variety of PEGeNB-X macromers have been synthesized and tested and found to have advantageous and unexpected properties when used to produce hydrogel materials via thiol-norbornene photo-gelation. For example, various PEGeNB-X hydrogels were discovered to degrade even faster than PEGeNB-CA without the inclusion of additional degradable macromer, some variants having degradation rates that were up to about 100 times faster than that with unconjugated PEGeNB-CA hydrogels. This represents a further significant improvement in the design and use of PEG-based hydrogels in tissue engineering applications. The hydrogel systems described herein are highly cytocompatible and support the growth of viable cells. Moreover, this secondary conjugation did not affect gelation efficiency and the shear moduli.

In addition to the methods described above, in other aspects, this disclosure provides macromers and also hydrogels produced using such macromers. In some embodiments, a macromer for producing a hydrogel comprises a multi-arm polyethylene glycol core having at least two polyethylene glycol domains, each polyethylene glycol domain connected through an ester linkage to a norbornene moiety, wherein at least one norbornene moiety of the macromer comprises a carboxyl group. In some embodiments, at least one of the polyethylene glycol domains comprises from about 10 to about 300 —CH2—CH2—O— units. In some embodiments, the multi-arm polyethylene glycol core has a number of polyethylene glycol domains selected from the group consisting of two, three, four and eight. In some embodiments, the macromer comprises at least one component having a structure as shown in Formula 1 above. In some embodiments, each of at least two norbornene moieties of the macromer comprises a carboxyl group. In some embodiments, a macromer for producing a hydrogel comprises a multi-arm polyethylene glycol core having at least two polyethylene glycol domains, each polyethylene glycol domain connected through an ester linkage to a norbornene moiety, wherein at least one norbornene moiety of the macromer is connected through a carboxamide linkage to a functional group. In some embodiments, the macromer comprises at least one component having a structure as shown in Formula 2 above. In some embodiments, the X of Formula 2 is selected from the group consisting of Formula 3. Formula 4, Formula 5, Formula 6, and Formula 7, shown above. In some embodiments, at least two norbornene moieties of the macromer are connected through a carboxamide linkage to a functional group.

In another aspect of the disclosure, there is provided a method for making a hydrogel that includes: (i) providing a first quantity of a PEGeNB-CA macromer, a PEGeNB-X macromer, a combination thereof or a combination of one or both of a PEGeNB-CA macromer and a PEGeNB-X macromer with one or more other PEGNB macromers; (ii) providing a second quantity of dithiols; (iii) mixing the first quantity of macromers with the second quantity of dithiols to provide a reaction mixture; and (iv) applying ultraviolet radiation to the reaction mixture to initiate a photo-gelation reaction to yield a hydrogel. In some embodiments, PEGeNB-CA and PEGeNB-X macromers are mixed together in various proportions ranging from 100:1 to 1:100 before initiating a photo-gelation reaction to yield a hydrogel. In other embodiments, PEGeNB-CA macromers and/or PEGeNB-X macromers are combined with one or more other PEGNB macromers before initiating a photo-gelation reaction to yield a hydrogel. In such embodiments, the PEGeNB-CA and/or PEGeNB-X macromer is present in the mixture in an any amount between 0% and 100% relative to the one or more other PEGNB macromers, In some embodiments, the hydrogel comprises an orthogonal thiol-norbornene hydrogel.

The proportions of the first quantity and the second quantity to one another may be determined based upon the stoichiometrie proportions of reactive groups necessary to optimize the reactivity of the reactants, and other practical considerations that are well within the purview of a person of ordinary skill in the art.

In some embodiments, the dithiol molecule comprises a multi-functional thiol. In some embodiments, the dithiol molecule comprises a member selected from the group consisting of 1,4-dithiothreitol, 4-arm thiolated PEG, a peptide that includes more than one cysteine residue, a natural or thiolated protein such as thiolated gelatin, thiolated hyaluronic acid, and thiolated collagens, and combinations thereof. In some embodiments, the dithiol molecule comprises dithiothreitol.

In some embodiments, the hydrogel has a hydrolytic degradation rate of from about 0.0045 to about 1.46 h⁻¹. In some embodiments, the hydrogel bas a hydrolytic degradation rate of from about 0.008 to about 1.46 h⁻¹. In some embodiments, the hydrogel has a hydrolytic degradation rate of from about 0.09 to about 1,46 h⁻¹. In some embodiments, the hydrolytie degradation rate is independent of pH.

In addition to the methods described above, in another aspect, the disclosure provides hydrogel materials produced as described herein. In some embodiments, the hydrogel materials are provided as bulk materials. In some embodiments, the hydrogel materials are contained within packages or containers. In some embodiments, the hydrogel materials are contained within packages or containers as a part of a kit that includes one more additional elements. In some embodiments, the hydrogel materials are contained within packages or containers with information in printed form or recorded in an electronic medium. In some embodiments, the information includes instructions for using the hydrogel materials. In some embodiments, the information includes a description of properties or characteristics of the hydrogel material contained within the package or container.

In other aspects of the disclosure, there are provided a wide variety of methods for using a hydrogel material made in accordance with this disclosure including, for example and without limitation, in biomaterials and tissue engineering applications. These hydrogel materials may be put to a wide variety of purposes and uses, including for example and without limitation, as a substrate for short-term 2D cell culturing, 3D in situ encapsulation of viable cells, controlled delivery of drugs and other bioactive materials, tissue engineering, tissue regeneration. creating micropores in a bulk polyethylene glycol-based hydrogel, creating a sacrificial material in a 3D bioprinted article, creating a sacrificial material in a photolithography-based biofabrication product, and creating a sacrificial bioink for extrusion-based or photolithography-based 3D bioproducts.

Potential uses of such PEG-based hydrogels include the use as templates for 3D culturing of cells, such as multipotent mesenchymal stem cells. Multipotent mesenchymal stem cells (MSCs) have high potential for many cell-based therapies as they can be isolated from multiple sources (e.g., bone marrow, umbilical cord, adipose tissue, etc.) and be readily differentiated into adipocytes, osteoblasts, and chondrocytes. MSCs can also undergo transdifferentiation into cardiomyocytes, neurons, and corneal epithelium cells. Additionally, studies have found that the secretory factors of MSCs can be utilized for regulating inflammatory and immune responses. Notably, MSC secretome can be manipulated by controlling matrix stiffness, viscoelasticity, and the presence of biological motifs (e.g., fibronectin). Many MSC-secreted factors, including transforming growth factor-β (TGF-β), insulin-like growth factor (IGF), and fibroblast growth factor-2 (FGF-2), have been shown to stimulate cell proliferation, migration, and differentiation. Furthermore, studies have found that, compared with 2D culture, MSCs cultured in 3D spheroids promoted cell proliferation, secretory properties, cell-cell interactions, and in vivo survival. In particular, a 2D surface un-naturally polarizes cells and lacks characteristics/architectures of a native cellular microenvironment needed to maintain the phenotype of the isolated cells. Studies have also revealed that assembling MSCs into multi-cellular spheroids increased the level of cell-cell interaction, leading to dramatically improved secretory properties and differentiation potential towards certain lineages (e.g., hepatocytes, osteoblasts, and neurons). MSCs have also been reported to exhibit anti-cancer effects via enhancing AKT signaling.

3D hydrogel scaffolds with physicochemically relevant properties have increasingly been used for MSC culture. Unless using hydrogel amenable to controllable degradation. chemically crosslinked hydrogels generally have nanometer-scale pores (or mesh) that will impede macromolecular diffusivity. Additionally, MSCs do not naturally form spheroids in 3D hydrogels. bence the encapsulated MSCs would remain disperse within the hydrogel matrix. The most efficient method to generate MSC clusters is by using well-plates with a hydrophobic surface and/or with special geometry (e.g., AGGREWELL™ plate). After assembly, MSC spheroids are collected and encapsulated in a hydrogel matrix to provide the cells with desired cell-matrix interactions. However, the handling of MSC sphernids may be challenging and the distribution of MSC spheroids within the bulk hydrogels may be non-uniform due to the settlement of the multicell spheroids. Furthermore, very few methods permit the generation of hollow cell spheres with a lumen structure, such as that found in ocular lens and lung alveoli. Therefore, a robust and powerful material engineering platform is needed to assist structured MSCs assembly in situ.

Hydrogels with macroscopic pores (i.e., macroporous hydrogels) are ideal for many biomedical applications owing to their improved transport properties and opportunity for enhanced cell-cell interactions within the matrix. Given the biphasic nature of the macroporous hydrogels, extracellular properties of the encapsulated cells could be decoupled from the bulk hydrogel matrix characteristics. As such, cellular assembly, attachment, and differentiation could be controlled by tuning extracellular properties independent of hydrogel stiffness. viscoelasticity, and cell adhesiveness,

A number of approaches bave been employed to fabricate macroporous hydrogels. including salt leaching and gas forming, which depend on embedding and removing porogens from the crosslinked hydrogels. While this approach is straightforward, commonly used porogens (e.g., NaCl crystals and CO₂) are not compatible with in situ cell encapsulation. Furthermore, post-synthesis cell seeding is required but the passive penetration of cells into the porous hydrogel may be challenging, resulting in non-uniform cell distribution. Alternatively, cell-laden microgel templating within a continuous matrix could directly introduce cells into a 3D environment and cell structures could be formed in situ. But sacrificial materials (e.g., pH, photo, enzyme or thermal sensitive) are either limited in cytocompatibility or their degradation products (e.g., gelatin-derived peptides) may alter stem cell signaling pathways. Even though gelatin and alginate based hydrogels/microgels offer good cytocompatibility to the encapsulated cells, their dissolution requires changing temperature (for gelatin) or using external molecular trigger (e.g., metal ion chelators).

Recently, microporous annealed particle hydrogels (MAP) have become an attractive strategy where MSCs were seeded and attached on the annealed microgel interfaces. However, current MAP scaffolds cannot support MSC assembly into spheroids. Alternatively. hydrogels susceptible to hydrolytic degradation could be barnessed for forming dissolvable microgels to template the otherwise nanoporous hydrogels into macroporous gels. For example, microgels crosslinked from poly(ethylene glycol) (PEG) diacrylate (PEGDA) and dithiol crosslinker have been templated as sacrificial phase. The ester bonds in crosslinked microgels are labile toward hydrolysis for fabricating macroporous hydrogels. However, conventional ester hydrolysis induced gel degradation is a slow process that precludes the applications of such a platform in sensitive cell candidates that rely on cell-cell interaction to survive and function.

In some embodiments, droplet-microfluidics may be employed to fabricate cell-laden dissolvable microgels with well-controlled size and degradation profiles. These cell-laden dissolvable microgels may then be loaded into another bulk hydrogel. Macroporous hydrogels may then be formed by allowing the microgels to dissolve, leaving behind the cells in the pores. By tuning cell adhesiveness of the continuous matrix, multi-cellular structures may be formed.

In embodiments, the multi-cellular structures may include pancreatic cancer cells COLO-357. lung epithelial cells A549, mouse MSCs, human MSCs, or a combination of two or more of these. Besides morphological tunability, different assembly structures may also affect MSC therapeutic potential. This effect may be investigated by analyzing phosphorylation of a panel of AKT signaling proteins or the secretion of growth factors and cytokines.

In some embodiments a hydrogel comprises a cross-linked network that is composed of: (i) a plurality of multiarm polyethylene glycol structures, each of said plurality of multiarm polyethylene glycol structures including at least two polyethyle glycol domains covalently bonded to one another directly or through a covalent connecting structure comprising one or more atoms: (ii) a plurality of norbornene domains; and (iii) a plurality of dithio-derived cross-linkers; wherein each of said plurality of polyethylene glycol domains is covalently bonded to one of said plurality of a norhornene domains via an ester linkage; wherein each of said plurality of norbornene domains includes a carboxyl group or a carboxamide linking a functional group to said porbornene domain; wherein each of said dithio-derived cross-linkers is covalently bonded to two of said plurality of norbornene domains; and wherein said hydrogel exhibits faster hydrolytic degradation compared to a hydrogel having the same structure except lacking the carboxyl group or carboxamide.

The processes and hydrogel materials in accordance with this disclosure also provide for the ability to tune hydrogel degradation kinetics. In one aspect of the disclosure, hydrogel materials are synthesized in a controlled manner to have pre-engineered hydrolytic degradation profiles tuned for a particular use within a range of desired properties. The profiles are tuned by selection of functional moieties within the macromers and/or by selection of various combinations of macromers and proportions of PEGeNB-CA and PEGeNB-X macromers within such combinations and/or by selection of dithiols having different properties for cross-linking the macromers to form the hydrogel materials. In some embodiments. a method for tuning hydrogel degradation kinetics includes: (i) mixing at least two norbornene-functionalized polyethylene macromers, at least one of the norbornene-functionalized polyethylene macromers selected from the group consisting of a PEGeNB-CA macromer and a PEGeNB-X macromer; (ii) controlling the proportions of the at least two norbornene-functionalized polyethylene macromers based on at least one desired hydrogel characteristic selected from the group consisting of cross-link density, hydrolytic degradation kinetics, shear modulus and swelling ratio; (iii) mixing the at least two norbornene-functionalized polyethylene macromers with a predetermined quantity of dithiol molecules to provide a reaction mixture; and (iv) applying ultraviolet radiation to the reaction mixture to initiate a photo-gelation reaction to yield a hydrogel having tuned degradation kinetics. In some embodiments, the method also includes selecting the dithiol molecules based on a functional characteristic of the dithiol molecules that modifies the degradation kinetics of the hydrogel.

Aspects of the present disclosure can be described as embodiments in any of the following enumerated clauses. It will be understood that any of the described embodiments can be used in connection with any other described embodiments to the extent that the embodiments do not contradict one another.

In an aspect, either alone or in combination with any other aspect, a method for synthesizing a norbornene-functionalized polyethylene glycol macromer, includes: providing a polyethylene glycol reactant comprising at least one polyethylene glycol domain with a terminal hydroxyl group; and reacting the polyethylene glycol reactant with carbic anhydride in the presence of a nucleophilic catalyst to yield a norbornene-functionalized polyethylene glycol macromer having an ester linkage between a polyethylene glycol domain of the macromer and a norbornene domain of the macromer, wherein the norbornene domain comprises a carboxyl group.

In an aspect, either alone or in combination with any other aspect, the polyethylene glycol reactant comprises a multi-arm polyethylene glycol molecule.

In an aspect, either alone or in combination with any other aspect, the polyethylene glycol reactant comprises a polyethylene glycol molecule selected from the group consisting of a 2-arm polyethylene glycol molecule, a 3-arm polyethylene glycol molecule, a 4-arm polyethylene glycol molecule, a 6-arm polyethylene glycol molecule, an 8-arm polyethylene glycol molecule and combinations thereof.

In an aspect, either alone or in combination with any other aspect, the nucleophilic catalyst comprises 4-dimethylaminopyridine.

In an aspect, either alone or in combination with any other aspect, the norbornene-functionalized polyethylene glycol macromer comprises a structure as shown in Formula 1 wherein n=from about 10 to about 300, and Ri comprises a member selected from the group consisting of a second arm of PEG, optionally additional arms of PEG, a macromolecule, a PEGylated inorganic compound that includes a tail of PEG, and combinations thereof.

In an aspect, either alone or in combination with any other aspect, the method further includes conjugating the carboxyl group of the norbornene domain with an amine-containing molecule to provide a modified macromer.

In an aspect, cither alone or in combination with any other aspect, the amine-containing molecule comprises a member selected from the group consisting of L-3,4-dihydroxyphenethylamine, tyramine, isopropylamine and combinations thereof.

In an aspect, either alone or in combination with any other aspect, the modified macromer comprises at least one component having a structure as shown in Formula 2 wherein n=from about 10 to about 300, X is an aromatic group or an aliphatic group, and R₁ comprises a member selected from the group consisting of a second arm of PEG, optionally additional arms of PEG, a macromolecule, a PEGylated inorganic compound that includes a tail of PEG, and combinations thereof.

In an aspect, either alone or in combination with any other aspect, X is selected from the group consisting of a functional group of Formula 3, a functional group of Formula 4, a functional group of Formula 5, a functional group of Formula 6, and a functional group of Formula 7.

In an aspect, either alone or in combination with any other aspect, a method for modifying a norbornene-functionalized polyethylene glycol macromer includes: providing a norbornene-functionalized polyethylene glycol macromer having an ester linkage between a polyethylene glycol domain of the macromer and a norbornene domain of the macromer, wherein the norbornene domain comprises a carboxyl group; and conjugating the carboxyl group of the norbornene domain with an amine-containing molecule to provide a modified macromer.

In an aspect, either alone or in combination with any other aspect, a macromer for producing a hydrogel includes a multi-arm polyethylene glycol core having at least two polyethylene glycol domains, each polyethylene glycol domain connected through an ester linkage to a norbornene moiety, wherein at least one norbornene moiety of the macromer comprises a carboxyl group.

In an aspect, cither alone or in combination with any other aspect, each of the at least two polyethylene glycol domains comprises from about 10 to about 300 —CH₂—CH₂—O— units.

In an aspect, either alone or in combination with any other aspect, the multi-arm polyethylene glycol core has a number of polyethylene glycol domains selected from the group consisting of two, three, four, six and eight.

In an aspect, either alone or in combination with any other aspect, the macromer comprises at least one component having a structure as shown in Formula 1 wherein n=from about 10 to about 300, and R₁ comprises a member selected from the group consisting of a second arm of PEG. optionally additional arms of PEG, a macromolecule, a PEGylated inorganic compound that includes a tail of PEG, and combinations thereof.

In an aspect, either alone or in combination with any other aspect, at least two norbornene moieties of the macromer comprise a carboxyl group.

In an aspect, either alone or in combination with any other aspect, a macromer for producing a hydrogel includes a multi-arm polyethylene glycol core having at least two polyethylene glycol domains, each polyethylene glycol domain connected through an ester linkage to a norbornene moiety, wherein at least one norbornene moiety of the macromer is connected through a carboxamide linkage to a functional group.

In an aspect. either alone or in combination with any other aspect, each of the at least two polyethylene glycol domains comprises from about 10 to about 300 —CH₂—CH₂—O— units.

In an aspect, either alone or in combination with any other aspect, the multi-arm polyethylene glycol core has a number of polyethylene glycol domains selected from the group consisting of two, three, four, six and eight.

In an aspect, either alone or in combination with any other aspect, the macromer comprises at least one component having a structure as shown in Formula 2 wherein n=from about 10 to about 300, X is an aromatic group or an aliphatic group, and Ri comprises a member selected from the group consisting of a second arm of PEO, optionally additional arms of PEG, a macromolecule, a PEGylated inorganic compound that includes a tail of PEG, and combinations thereof.

In an aspect, either alone or in combination with any other aspect, X is selected from the group consisting of a functional group of Formula 3, a functional group of Formula 4, a functional group of Formula 5, a functional group of Formula 6, and a functional group of Formula 7.

In an aspect, either alone or in combination with any other aspect, at least two norbornene moieties of the macromer are connected through a carboxamide linkage to a functional group.

In an aspect, either alone or in combination with any other aspect, A method for making a hydrogel includes providing a first quantity of macromers comprising a macromer selected from the group consisting of the macromer of any preceding aspect and combinations thereof; providing a second quantity of dithiol molecules; mixing the first quantity of macromers with the second quantity of dithiol molecules to provide a reaction mixture; and applying ultraviolet radiation to the reaction mixture to initiate a photo-gelation reaction to yield a hydrogel.

In an aspect, either alone or in combination with any other aspect, the dithiol molecule is operable as a hydrogel cross-linker.

In an aspect, either alone or in combination with any other aspect, the dithiol molecule is selected from the group consisting of 1,4-dithiothreitol, 4-arm thiolated PEG, a peptide that includes more than one cysteine residue, a natural or thiolated protein such as thiolated gelatin. thiolated hyaluronic acid, and thiolated collagens, and combinations thereof.

In an aspect, either alone or in combination with any other aspect, the dithiol molecule comprises dithiothreitol.

In an aspect, either alone or in combination with any other aspect, the hydrogel comprises an orthogonal thiol-norbornene hydrogel.

In an aspect, either alone or in combination with any other aspect, the hydrogel has a hydrolytic degradation rate of from about 0.0045 to about 1.46 h⁻¹.

In an aspect, either alone or in combination with any other aspect, the hydrolytic degradation rate is independent of pH.

In an aspect, either alone or in combination with any other aspect, a hydrogel may be produced according to any preceding aspect.

In an aspect, either alone or in combination with any other aspect, a hydrogel includes a cross-linked network composed of a plurality of multiarm polyethylene glycol structures, each of said plurality of multiarm polyethylene glycol structures including at least two polyethyle glycol domains covalently bonded to one another directly or through a covalent connecting structure comprising one or more atoms; a plurality of norbornene domains; and a plurality of dithio-derived cross-linkers. Each of said plurality of polyethylene glycol domains is covalently bonded to one of said plurality of a norbornene domains via an ester linkage. Each of said plurality of norbornene domains includes a carboxyl group or a carboxamide linking a functional group to said norbomene domain. Each of said dithio-derived cross-linkers is covalently bonded to two of said plurality of norbornene domains. Said hydrogel exhibits faster hydrolytic degradation compared to a hydrogel having the same structure except lacking the carboxyl group or carboxamide.

In an aspect, either alone or in combination with any other aspect, the hydrogel may be used for a purpose selected from the group consisting of as a substrate for short-term 2D cell culturing, 3D in situ encapsulation of viable cells. controlled delivery of drugs and other bioactive materials. tissue engineering, tissue regeneration, creating micropores in a bulk polyethylene glycol-based hydrogel, creating a sacrificial material in a 3D bioprinted article, creating a sacrificial material in a photolithography-based biofabrication product, and creating a sacrificial bioink for extrusion-based or photolithography-based 3D bioproducts.

In an aspect, either alone or in combination with any other aspect, a method for tuning hydrogel degradation kinetics includes mixing at least two norbornene-functionalized polyethylene macromers, at least one of the norbornene-functionalized polyethylene macromers selected from a macromer of any preceding aspect; controlling the proportions of the at least two porbornene-functionalized polyethylene macromers based on at least one desired bydrogel characteristic selected from the group consisting of cross-link density, hydrolytic degradation kinetics, shear modulus and swelling ratio; mixing the at least two norbornene-functionalized polyethylene macromers with a predetermined quantity of dithiol molecules to provide a reaction mixture; and applying ultraviolet radiation to the reaction mixture to initiate a photo-gelation reaction to yield a hydrogel having tuned degradation kinetics.

In an aspect, either alone or in combination with any other aspect, a method for forming a macroporous hydrogel, the macroporous hydrogel comprising at least one cell, includes: mixing a hydrogel-forming solution with a media containing the at least one cell, the hydrogel-forming solution comprising a crosslinkable material: crosslinking at least a portion of the crosslinkable material of the hydrogel-forming solution, thereby forming a microgel comprising the at least one cell and a sacrificial material; dispersing the microgel in a bulk hydrogel; and removing the sacrificial material, thereby forming the macroporous hydrogel.

In an aspect, either alone or in combination with any other aspect, the at least one cell comprises a plurality of cells, and at least a portion of the plurality of cells accumulate into a multicellular cluster in at least one pore of the macroporous hydrogel.

In an aspect, either alone or in combination with any other aspect, the multicellular cluster is in a spheroidal structure.

In an aspect, either alone or in combination with any other aspect, the spheroidal structure is a hollow spheroid.

In an aspect, either alone or in combination with any other aspect. the spheroidal structure is a solid spheroid.

In an aspect, either alone or in combination with any other aspect, the at least one cell comprises at least one mesenchymal stem cell.

In an aspect, either alone or in combination with any other aspect, the at least one cell comprises at least one cell selected from the group consisting of pancreatic cancer cells COLO-357, lung epithelial cells A549, mouse MSCs, human MSCs, or a combination of two or more of these.

In an aspect, either alone or in combination with any other aspect, the crosslinkable material comprises at least one component having a structure as shown in Formula 2 wherein n=from about 10 to about 300. X is an aromatic group or an aliphatic group, and R; comprises a member selected from the group consisting of a second arm of PEG, optionally additional arms of PEG, a macromolecule, a PEGylated inorganic compound that includes a tail of PEG, and combinations thereof.

In an aspect, either alone or in combination with any other aspect, X is selected from the group consisting of a functional group of Formula 3, a functional group of Formula 4, a functional group of Formula 5, a functional group of Formula 6, and a functional group of Formula 7.

In an aspect, either alone or in combination with any other aspect, X is a functional group of Formula 3.

Further reference is made to the following experimental examples.

### EXAMPLES

The following examples are given for the purpose of illustrating various embodiments of the invention and are not meant to limit the present invention in any fashion. The present examples, along with the methods described herein are presently representative of preferred embodiments, are provided only as examples, and are not intended as limitations on the scope of the invention, Changes therein and other uses which are encompassed within the spirit of the invention as defined by the scope of the claims will occur to those skilled in the art.

### Examples 1

Carbic anhydride-based PEGeNB synthesis and PEGeNB-CA characterization compared to conventional, To functionalize hydrolytically labile PEGNB (FIG. 1, Panel A, X=H) through Steglich esterification, the conventional synthesis approach requires that 5-norbornene-2-carboxylic acid (NB-acid) be activated with N,N′-dicyclohexylcarbodiimide (DCC) to form the O-acylisourea intermediate (FIG. 1, Panel A, 1). The intermediate is filtered into PEG-hydroxyls in the presence of pyridine and 4-dimethylaminopyridine (DMAP), followed by an overnight reaction and precipitation in cold ether. To maximize the degree of substitution (DS), multiple efforts were developed, such as extended refluxing PEG in toluene to remove excess bond water, blanketing the reaction with inert gas, conducting back-to-back reactions, and limiting the reaction to smaller batches (<5 g). Unfortunately, none of these lengthy and laborious steps (approximately 5 days) eliminates the pungent and extremely unpleasant odor of NB-acid. Stringent containment efforts are taken during and post-synthesis to minimize the unpleasant odor of NB-acid. While PEGNB macromers are commercially available, they are expensive and, hence, not a sustainable option. These disadvantages significantly limit the widespread usage of this powerful and diverse macromer.

Synthesis reaction protocols using carbic anhydride (CA) described here circumvent the cumbersome steps in the conventional PEGNB synthesis. CA is an odorless porbornene derivative that includes a diacid anhydride group (FIG. 1, Panel A, II). Depicted in FIG. 1 is a user-friendly, efficient CA-based synthesis protocol for introducing norbornene to multiarm PEG-hydroxyls. FIG. 1 shows synthesis and characterization data for PEGNBcA hydrogels. Panel A depicts reaction and purification routes for the synthesis of norbornene-functionalized PEG, by both a conventional approach and by the process described herein. Panel B depicts the light (hv) and radical initiated thiol-norbornene gelation by lithium phenyl-2,4,6-trimethylbenzoylphosphinate (LAP). Exemplary conditions for the reaction depicted in Panel B include 1 mM LAP, 2 min exposure to light at 365 nm and5 mW/cm². Panel C provides the in situ photorheometry of 2.5 wt % PEGNBca with DTT (R=1). Panel D provides the shear moduli of hydrogels cross-linked by PEGNB or PEGNBca with DTT.

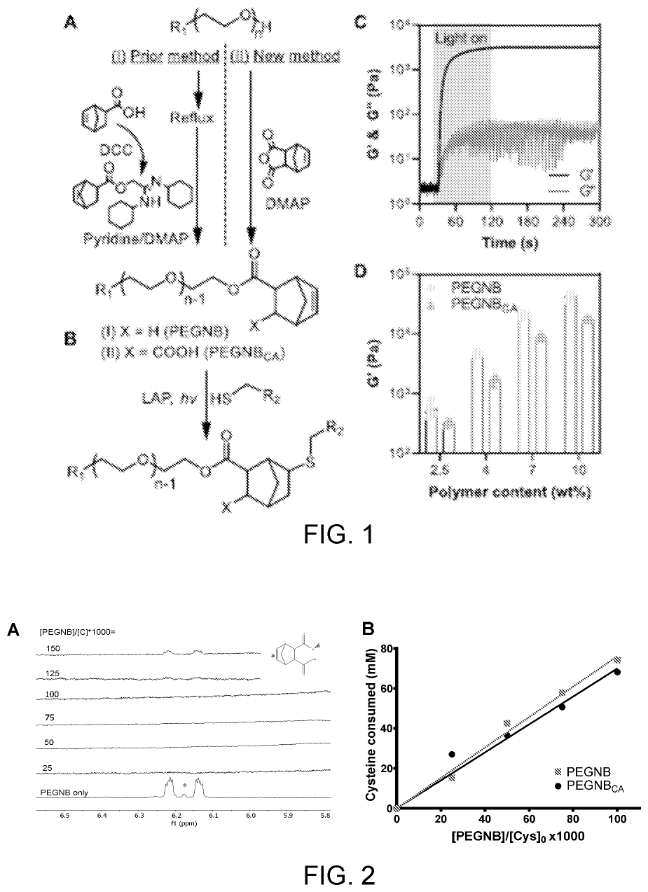

The reaction based on CA was catalyzed by DMAP in tetrahydrofuran (THE) (FIG. 1, Panel A, II), affording a macromer PEGeNB-CA (FIG. 1, Panel A, X═COOH). No odor containment effort was needed for the PEGeNB-CA synthesis, and the method shortened the overall synthesis time from about 5 days to about 2 days. High NB substitution in PEGeNB-CA was verified via quantifying thiol consumption using thiol-norbornene reaction with cysteine (Cys). FIG. 2 provides ¹H NMR spectra (Panel A) and quantification of residual Cys using Ellman's assay. The initial Cys concentration ([Cys]o) was 103 mM, and the degree of substitution of the PEGeNB-CA DSPEGNECA) was 87%. Morcover, the PEGeNB-CA macromer contains an additional carboxylic acid moiety, which can be exploited for subsequent conjugation of functional groups via standard carbodiimide chemistry.

Thiol-norbornene photogelation, as shown in Panel B of FIG. 1, was tested using the PEGeNB-CA and dithiothreitol (DTT) at a stoichiometric ratio (i.e., R_{thiol/ene}=1). The gelation kinetics as evaluated by in situ photorheometry revealed a rapid gel point (about 10 s) typical to the UV-initiated thiol-norbornene gelation (FIG. 1, Panel C). However, compared with hydrogels cross-linked by PEGNB prepared by conventional processes, the shear moduli (G′) of the hydrogels cross-linked by PEGeNB-CA were consistently lower at equivalent polymer contents, as shown by Panel D of FIG. 1, suggesting a lower reaction efficiency of PEGeNB-CA toward thiols. The reduced thiol-norbornene reaction efficiency of PEGeNB-CA may be a result from the additional carboxylic acid group that reduces the local pH value. Nonetheless, this carbic anhydride protocol offers a greener and more user-friendly route to the synthesis of norbornene-functionalized PEG for orthogonal thio-norbornene hydrogel cross-linking, and for other uses.

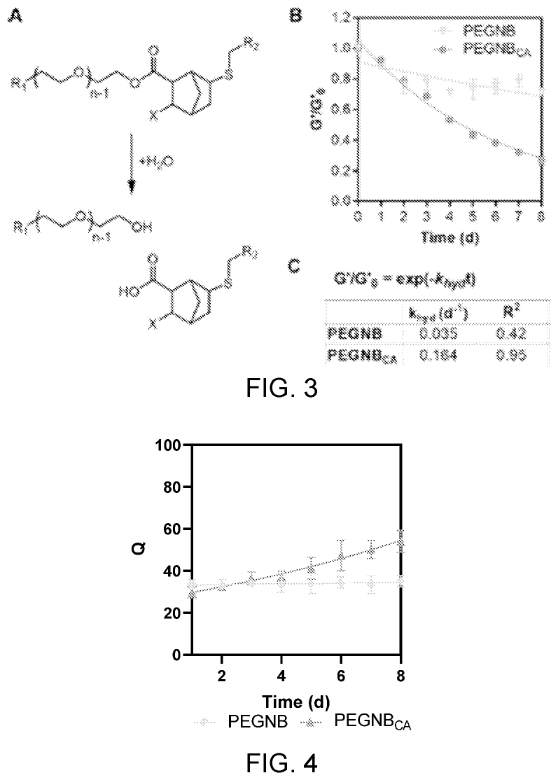

FIG. 3 depicts hydrolytic degradation of PEGNM and PEGNBex hydrogels. Panel A is a schematic of ester bond hydrolysis. Panel B provides hydrogel shear moduli changes (G′/G′a) as a function of time. Panel C provides parameters of pseudo-first order hydrolytic degradation kinetics. Ester-lined PEGNB hydrogels (i.e., PEGeNB) prepared by conventional processes are known to undergo slow and predictable hydrolytic degradation. Indeed, 4 wt % of PEGeNB-DTT hydrogels degraded partially (G′/G's of about 25%) after 8 days of incubation (FIG. 3, Panel B).

Hydrogels cross-linked by the PEGeNB-CA macromer degraded much faster than the PEGeNB counterpart prepared by conventional processes (FIG. 3, Panel B and FIG. 4). FIG. 4 depicts the degradation of PEGNB and PEGNBex hydrogels at 37° C. by swelling ratio.

After further analysis of the hydrolytic degradation kinetics with pseudo-first order degradation kinetics (G′/G′₀=exp (−K_{hydt})), a hydrolysis kinetic rate constant (k_{hyd}) of 0.035^{d-1} and 0.164 d⁻¹ was obtained for gels made using previously described PEGeNB and PEGeNB-CA of the present disclosure, respectively (FIG. 3, Panel C). Thiol-norbornene hydrogels prepared in accordance with conventional processes did not degrade sufficiently in the first few days, the line fit for PEGeNB gel was not ideal (R²=0.42); however, the k_{hyd} value (0.035 d⁻¹) was within the range of previous results with longer term degradation (i.e., k_{hyd}=0.02-0.07 d⁻¹). The R² value for curve fitting of PEGeNB-CA degradation, on the other hand, reached over 0.95, indicating that the degradation of PEGeNB-CA gels was a result of ester bond hydrolysis. Compared with previously described PEGeNB-DTT hydrogels. PEGeNB-CA-DTT hydrogels degraded much faster and reached complete degradation within 2 weeks (data not shown). In addition to the moduli change, the degradation was also tracked by the swelling ratio (Q; FIG. 4). In particular, the swelling ratios of previously described PEGeNB-DTT hydrogels and PEGeNB-CA-DTT hydrogels were comparable (Q of about 30) on day 1. However, the swelling ratio of PEGeNB-CA-DTT hydrogels continuously increased while that of previously described PEGeNB-DTT hydrogels remained relatively unchanged during the 8 days of incubation course. The accelerated hydrolysis of PEGeNB-CA hydrogels was likely due to the presence of an additional carboxylic acid group that destabilizes the ester bonds. The faster degradation kinetics of PEGeNB-CA hydrogels presents opportunities for many applications in which controllable and/or tunable hydrogel degradation is desired.

PEG-based thiol-norbornene hydrogels are known for their high cytocompatibility for in situ cell encapsulation. The synthesized PEGeNB-CA hydrogels also showed excellent cytocompatibility, as exemplified by the encapsulation of human induced pluripotent stem cells (hiPSC), dental pulp stem cells (DPSC). and PANC-1 human pancreatic cancer cells. The live/dead staining and confocal imaging of cell-laden PEGNBcs peptide hydrogels at Day I and at Day 7 is shown in FIG. 5. Notably, FIG. 5 shows that a large number of cells remain living on Day 7.

All hydrogels were cross-lined by matrix metalloproteinase (MMP)-sensitive peptide linker (i.e., SEQ ID NO: 1 [KCGPQG*IWGQCK, *cleavage site]) to permit cell-mediated matrix remodeling and immobilized with ImM RGDS peptide to support cell survival. Live/Dead staining results showed that very limited number of dead cells were visible for all cell types following encapsulation. After 7 days of in vitro culture, biPSC and PANC-1 cells proliferated to form multicellular spheroids, while DPSC exhibited spreading morphology, indicating extensive cell-mediated matrix cleavage. These results establish that PEGeNB-CA hydrogels are an attractive alternative material for in sito encapsulation of a variety of cells.

### Example 2

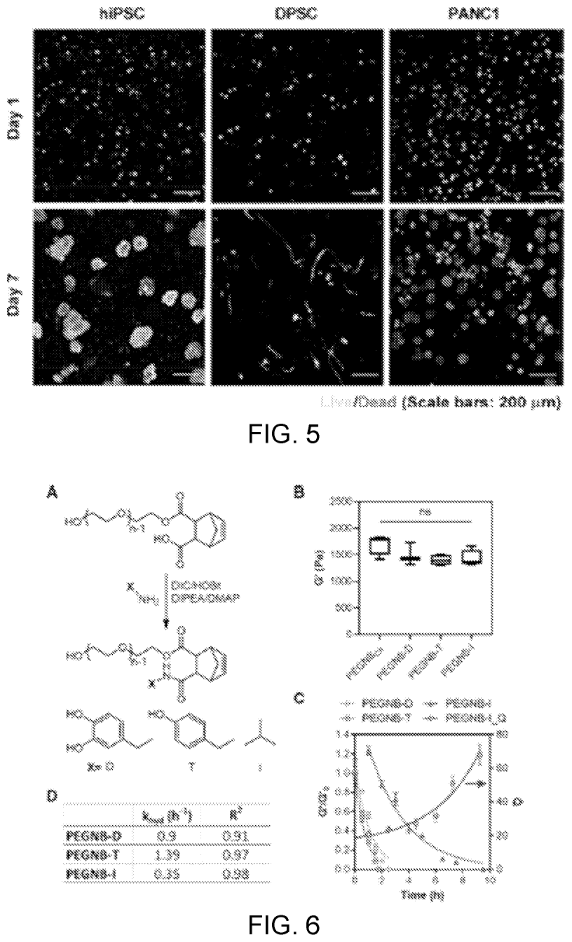

Secondary conjugation of carboxylic acid moiety of a PEGeNB-CA macromer. In addition to demonstrating an improvement in synthesis procedures and high cytocompatibility, the PEGeNB-CA macromer affords a carboxylic acid moiety amenable for secondary conjugation with amine-bearing molecules (FIG. 6, Panel A, PEGNB-X). FIG. 6 shows the cross-linking and degradation of PEGNB, PEGNB_{CA}, and PEGNB-X hydrogels. Panel A is a schematic of PEGNB-X synthesis. Panel B provides the initial shear moduli of hydrogels cross-linked by 4 wt % PEGNB_{CA}, PEGNB-D, PEGNB-T, and PEGNB-I with DTT. Panel C provides the degradation of PEGNB-X hydrogels at 37° C. by shear moduli changes (G′/G′₀) and swelling ratio (Q). The curve fittings represent pseudo first-order ester hydrolysis kinetics. Panel D provides the parameters of these pseudo first-order hydrolytic degradation kinetics. All hydrogels were cross-linked by DTT (R=1). Exemplary conditions for the cross-linking include 1 mM LAP, 2 min exposure to light at 365 nm and 5 mW/cm².

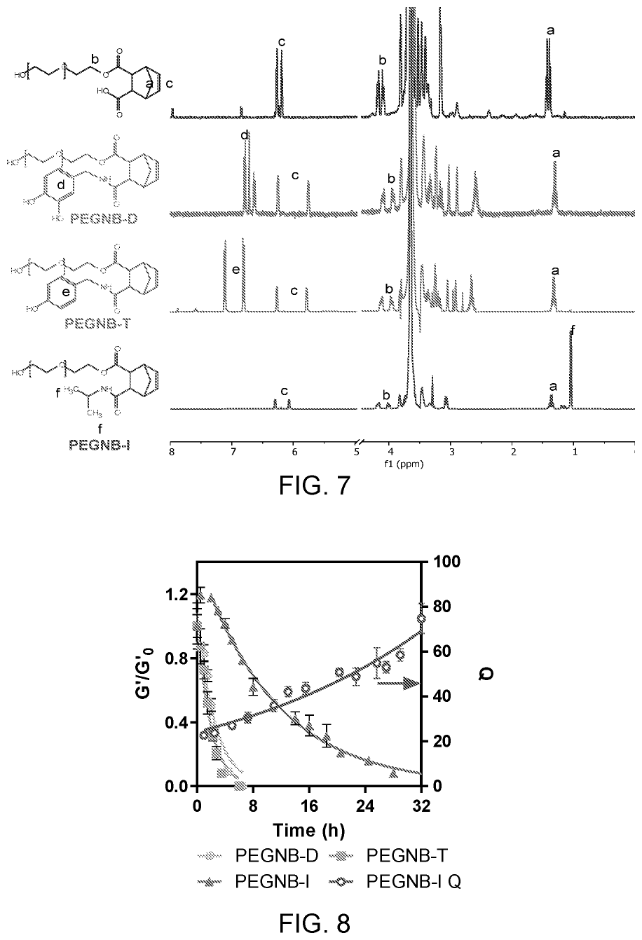

As described further below, the conversion of the X conjugates (e.g., L-dopamine (D), tyramine (T), or isopropylamine (I)) reached 83-100% depending on the reactant, as calculated from ¹H NMR spectra. FIG. 7 provides ¹H NMR spectra of PEGNB_{CA}, PEGNB-D), PEGNB-T, and PEGNB-I. The conversion of PEGNB-D, PEGNB-T. and PEGNB-I with respect to the norbornene group was 100%, 97%, and 83% respectively. These demonstrated X conjugates did not affect cross-linking efficiency of PEGeNB-X/DTT hydrogels, as demonstrated by G′₀ comparable to that of the PEGeNB-CA/DTT hydrogels (FIG. 6, Panel B). As discussed further below, it was discovered that the ester linkage in PEGeNB waas further weakened by the second conjugation. After overnight swelling at 37° C., all PEGeNB-X/DTT hydrogels completely degraded, suggesting that the secondary conjugation substantially decreased the stability of the ester bonds.

The degradation rate of PEGeNB-D, PEGeNB-T and PEGeNB-I hydrogels (FIG. 6, Panel C) was determined. At 37° C., PEGeNB-D and PEGeNB-T hydrogels fully degraded within 2 hours, while PEGeNB-I hydrogels degraded in about 10 hours (FIG. 6, Panel C). It is worth noting that the three sets of gels were cross-linked to have similar initial shear moduli (G′s of about 1.5 kPA, FIG. 6, Panel B). PEGeNB-D and PEGeNB-T degraded extremely fast, exhibiting degradation rates from about SO to about 200 times faster than that of PEGeNB-CA hydrogels (FIG. 3. Panel C). Interestingly, compared with the T or D conjugate. I conjugation slowed gel degradation considerably (FIG. 6, Panel C). Upon measuring swelling ratios (Q) to gain insight into their degradation behavior, it was observed that there was a limited initial swelling in the first 2 hours, after which the welling increased substantially (FIG. 6, Panel C). The limited initial swelling and increase in the initial G′ could be attributed to the hydrophobic interactions between isopropyl groups. Nonetheless, the degradation of PEGeNB-I hydrogels could still be described by pseudo-first order degradation kinetics (FIG. 6, Panel D). Furthermore, the hydrolytic degradation of PEGeNB-X hydrogels appeared to be temperature-dependent and the degradation rates were reduced at room temperature. FIG. & provides the degradation of PEGNB-X bydrogels at room temperature. Table I provides parameters of pseudo-first order ester hydrolysis kinetics of PEGNB-X hydrogels at room temperature, in accordance with FIG. 8.

TABLE 1

| PEGNB-X | PEGNB-D | PEGNB-T | PEGNB-I |
|---|---|---|---|
| k_{hyd} (h⁻¹) | 0.39 | 0.52 | 0.09 |
| R² | 0.93 | 0.95 | 0.98 |

### Example 3

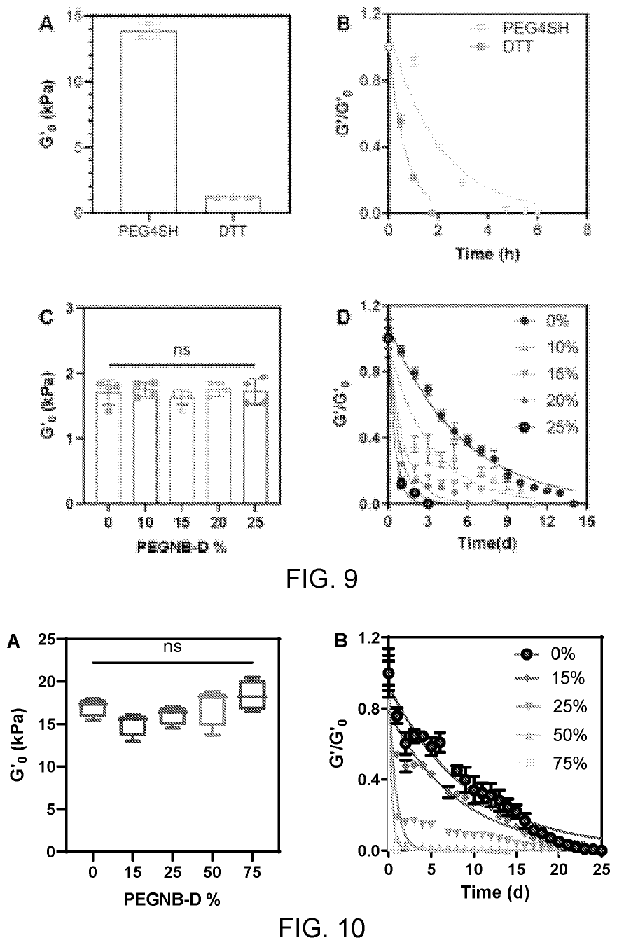

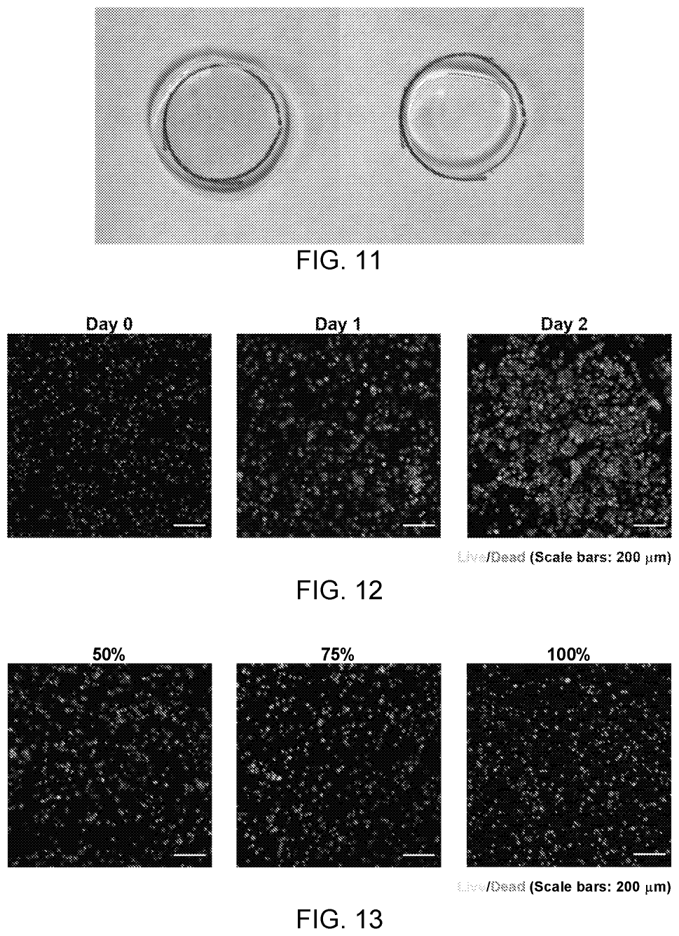

Tuning of hydrogel hydrolytic degradation kinetics. The facile cross-linking and rapid degradation of the PEGeNB-X hydrogels offer an opportunity to tune hydrogel degradation precisely by cross-linking with different cross-linkers and mixing PEGeNB-X with PEGeNB-CA while fixing the total PEGNB content. FIG. 9 demonstrates the control of degradation of PEGNB bydrogels via cross-linking with various cross-linkers and mixing in PEGNB-D. Panel A provides the initial shear moduli of 4 wt % PEGNB-D cross-linked with DTT and 4 arms-thiolated PEG (PEO4SH). Panel B provides the degradation of 4 wt % PEGNB-D cross-linked with DTT and 4 arms-thiolated PEG (PEGASH). Panel C provides the initial shear moduli of soft (4 wt %) PEGNBCA/PEGNB-D-DTT-D hydrogels with variable PEGNB-D content. Panel D provides the degradation of soft (4 wt %) PEGNBc//PEGNB-D-DTT-D hydrogels with variable PEGNB-D content. Table 2 provides parameters of pseudo-first order ester hydrolysis kinetics of 4 wt % PEGNB_{CA}/PEGNB-D hydrogels at 37° C., in accordance with FIG. 9, Panels C and D.

TABLE 2

| PEGNB-D | k_{hyd} | |
|---|---|---|
| wt % | (d⁻¹) | R² |
| 0 | 0.19 | 0.97 |
| 10 | 0.31 | 0.92 |
| 15 | 0.82 | 0.92 |
| 20 | 1.19 | 0.96 |
| 25 | 2.00 | 0.98 |

It is well-known that thiol-ene hydrogel cross-linking efficiency and hydrolytic degradation kinetics are both significantly affected by the functionality and structure of the cross-linkers. To demonstrate this, the cross-linking and degradation of PEGeNB-D hydrogels cross-linked by either tetra-functional (i.e., PEG4SH) or bifunctional thiol (i.e., DTT) was compared. At an identical PEGeNB-D macromer content (i.e., 4 wt %), hydrogels cross-linked by PEG4SH were considerably stiffer (G′₀ of about (4 kPa) than that formed with DTT (G′a of about 1.6 kPa, FIG. 9, Panel A). While these two sets of gels were cross-linked with an identical amount of norbornene ester bonds, the degradation kinetics were significantly different, with PEG4SH-cross-linked gels degrading roughly 3 times slower than that DTT-cross-linked gels (FIG. 9, Panel B). Table 3 provides parameters of pseudo-first order ester hydrolysis kinetics of PEGNB-D cross-linked with DTT and PEG4SH hydrogels at 37° C., in accordance with FIG. 9, Panels A and B. These results implied that the degradation of the hydrogels was primarily governed by the bulk hydrogel properties (i.e., swelling, cross-linking density), rather than by the kinetics of ester bond hydrolysis.

TABLE 3

| Cross- | | |
|---|---|---|
| linker | DTT | PEG4SH |
| k_{hyd} (h⁻¹) | 1.46 | 0.49 |
| R² | 0.97 | 0.91 |

In addition to tuning hydrogel degradation by varying the functionality of the cross-linker, the degradation can be further regulated by mixing PEGeNB-D with PEGeNB-CA at different ratios during hydrogel fabrication. At a fixed total PEGNB content (e.g., 4 or 10 wt %). the inclusion of PEGeNB-D did not alter hydrogel initial cross-linking (G′₀ of about 1.6 and 17 kPa for 4 wt % (soft) and 10 wt % (stiff) hydrogels, respectively, as shown in FIG. 9, Panel C, and FIG. 10). FIG. 10 provides the initial shear moduli (Panel Aj and the degradation (Panel B) of 10 w(%) PEONB_{CA}/PEGNB-D-DTT hydrogels with varying PEGNB-D content. Table 4 provides parameters of pseudo-first order ester hydrolysis kinetics of 10 wt % PEGNB_{CA}/PEGNB-D hydrogels at 37° ° C., in accordance with FIG. 10.

TABLE 4

| PEGNB-D | k_{hyd} | |
|---|---|---|
| wt % | (d⁻¹) | R² |
| 0 | 0.11 | 0.95 |
| 15 | 0.12 | 0.91 |
| 25 | 1.23 | 0.89 |
| 50 | 2.68 | 0.99 |

However, the degradation kinetics were decoupled from the degree of network connectivity (FIG. 9, Panel D, and FIG. 10, Panel B). For soft hydrogels, the degradation period ranged from about 3 to about 14 days (FIG. 9, Panel B). Notably, there was a significant drop in G′ carly on (1-2 days) when PEGeNB-D was included. For soft hydrogels incorporated with more than 25% PEGeNB-D, complete gel degradation occurred overnight (data not shown). These results implied that the rapid ester hydrolysis of PEGeNB-D quickly loosened the hydrogel network. leading to accelerated degradation in the nearby PEGeNB-CA macromer. Similar degradation was observed in stiff hydrogels. With these highly cross-linked networks, the degradation was further prolonged to 25 days (FIG. 10, Panel B). Interestingly, stiff hydrogels still completed degradation overnight when PEGeNB-D was higher than 50%. During this extended incubation, hydrogels notably swelled and discolored, likely due to oxidation of the residual dopamine. FIG. IT shows a 10 wt % PEGNB-D 25% hydrogel on Day 0 (left) and Day 7 (right). The circle diameter is 9 mm.

The cytocompatibillty of soft PEGeNB-CA/PEGeNB-D hydrogels at different PEGeNB-D and PEGeNB-CA ratios were further demonstrated by encapsulation of PANC-1 cells. FIG. 12 provides live/dead staining and confocal imaging of cell-laden 25%/75% PEGNB-D/PEGNBex-peptide hydrogels at Day 0, Day 1, and Day 2. FIG. 13 provides live/dead staining and confocal imaging of cell-laden PEGNB-D/PEGNB_{CA}-peptide hydrogels at a 50%, 75%, and 100% PEGNB-D ratio in the composites.

It is worth noting that the hydrogels with higher than 25% PEGeNB-D degraded overnight following encapsulation. Hence, cell viability staining was performed right after the encapsulation. For 25% PEGeNB-D hydrogels, the cell viability was tracked over 3 days as the gels degraded in a slower rate. In all conditions, PEGeNB-D hydrogels demonstrated exceptional cytocompatibility. Overall, this study establishes that it is possible to decouple the initial hydrogel moduli with their degradation rate, a unique feature not casily achievable by prior covalent hydrogels.

### Example 4

### Materials and Methods

### Materials

PEG-OH (8-arm, 20 kDa, JenKem), carbic anhydride (Sigma-Aldrich), 4-dimethylaminopyridine (DMAP, Alfa Aesar), dopamine hydrochloride (Sigma-Aldrich), tyramine hydrochloride (Chem-Impex), isopropylamine (TCI chemicals), N,N′-Diisopropylcarbodiimide (DIC, Chem-Impex), hydroxybenzotriazole (HOBt, ≥20 wt % water, Oakwood Chemical), N,N′-diisopropylethylamine (DIPEA, TCI chemicals), cysteine (Sigma-Aldrich), and lithium phenyl-2,4,6-trimethylbenzoylphosphinate (LAP, ≥95%, Sigma-Aldrich), dithiothreitol (DTT, Fisher), 4-arms thiolated PEG (PEG4SH, 10 kDa, Laysan Bio), CRGDS (GenScript), CGPQG*IWGQC a matrix metalloproteinase-sensitive peptide (*cleavage site, GenScript), 5,5 -dithiobis-(2-nitrobenzoic acid) (Ellman's reagent, Life Technologies), anhydrous tetrahydrofuran (THF, Fisher), anhydrous dimethylformamide (DMF, amine-free, Fisher), dichloromethane (DCM, Fisher), methanol (Sigma-Aldrich) and diethyl ether (Fisher) were used as received.

### PEGNBCA Synthesis

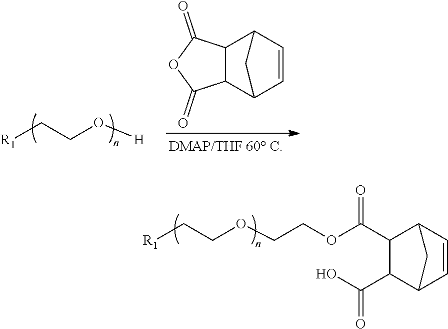

10 g PEG-OH (hydroxyl group 0.4 mmol), 3.28 g carbic anhydride (2 mmol, S equiv.), 0.49 g DMAP (0.4 mmol, 1 equiv.), and 83 mL anhydrous THE were charged in a round-bottom flask equipped with a stir bar. The reaction was conducted at 60° C. After 12 h, another portion of carbic anhydride and DMAP were added into the reaction. The reaction was continued for another 24 h. The product was retrieved via precipitation through diethyl ether twice and subsequently dried in vacuum. The conventional PEONB was synthesized according to Shih and Lin.

### Norbornene Substitution (NS) Ratio Determination

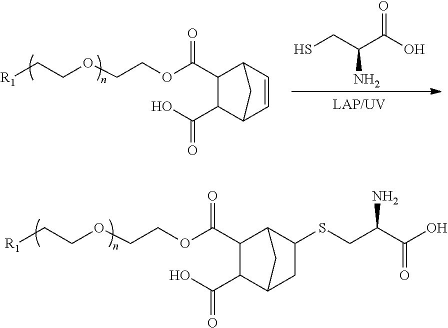

The norbornene degree of substitution (NS) ratio was characterized by photo-activated thiol-ene reaction described as follows: the predetermined amounts of PEGNB, cysteine (Cys) and LAP were mixed in phosphate buffer solution. The final concentration of cysteine and LAP was 100 mM and 2 mM, respectively. The solution was exposed under UV light (λ=365 nm) for 5 min. The PEGNB-to-cysteine ratio ([PEGNB]/[Cys]) was controlled between 0.05-0.1 since the double bond from NB was not fully consumed at higher ratios (FIG. 2). After being diluted 100 times, the solution was reacted with Ellman's reagent to determine the residual cysteine amount.

### PEGNB-X Synthesis

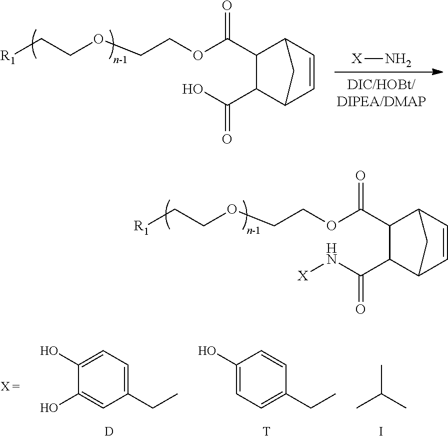

The method to conjugate amines with PEGNBea was adopted from Zhang et al. To synthesize PEGNBca derivatives (PEGNB-R), 1 g PEGNB_{CA}(carboxylic group 380 pmol), 148.8 μL DIC (950 μmol, 2.5 equiv.), 160 mg HOBt (950 μmol, 2.5 equiv.) and 10 mL amine-free DMF were charged into a reaction vial equipped with a stir bar. The activation of the acid group was blanketed under nitrogen gas for 2 h in dark. Designated amount of amines (180 mg dopamine bydrochloride, 165 mg tyramine hydrochloride, or 77.8 μL isopropylamine, 950 μmol, 2.5 equiv.), 165.4 μL DIPEA (950 μmol, 2.5 cquiv.), and 23 mg DMAP (190 μmol, 0.5 equiv.) were dissolved in 1 mL amine-free DMF and then added into the reaction. The reaction was continued overnight. The product was dialyzed in methanol for one day and dried out in vacuum. The product was characterized by ¹H NMR spectra (Bruker 500 MHz), as shown in FIG. 7.

### Hydrogel Fabrication

For rheological testing, predetermined amount of PEGNB, PEGNB_{CA}or PEGNB-Xs and DTT precursor solutions were mixed in phosphate buffer solution (PBS). 45 μl precursor solution was deposited between glass slides with 1 mm spacer and cross-linked by LAP under UV light (λ=365 nm, 5 mW/cm²) to afford hydrogel. The formulation details used in each figure are summarized in Table 5. The 0 h rheological results of PEGNB-X hydrogels shown in FIG. 6, FIG. 9, and FIG. 10 were acquired right after cured. Hydrogels were incubated in PBS at 37° C. between all experiments except in FIG. 8, which was kept at room temperature. Except the in situ acquisition in FIG. 1, Panel B, the dynamic shear rheology measurement was conducted by Malvern Bohlin CVO 100 rheometer with 8 mm parallel plates. The shear modulus was determined by strain sweep between strain of 0.1 and 5% at 1 Hz with a gap size of 700 μm. For the in-situ gelation acquisition in FIG. 1. Panel B, 100 μl. precursor solution was deposited between 25 mm parallel plates with a gap size of 90 μm. Modulus data were acquired at 1 Hz with UV light (λ=365 nm, 5 mW/cm²) shed between t=20-140 s for a total 5 min acquisition period. All results were averaged from 3 replicates.

TABLE 5

| | | PEGNB. | | | | | |
|---|---|---|---|---|---|---|---|
| | | PEGNB_{CA}, or | | | | | UV |
| FIG. | Norbomene | PEGNB-R | Cross- | Thiol/ene | [CRGDS] | [LAP] | exposure |
| (Panel) | source | (wt %) | linker | ratio | (mM) | (mM) | time (min) |
| 1 (B) | PEGNB_{CA} | 2.5 | DTT | 1 | 0 | 1 | 2 |
| 1 (C) | PEGNB_{CA} | varies | DTT | 1 | 0 | 1 | 2 |
| 1 (C) | PEGNB | varies | DTT | 1 | 0 | 1 | 2 |
| 3 (B), 8 | PEGNB_{CA} | 4 | DTT | 1 | 0 | 1 | 2 |
| 3 (B), 8 | PEGNB | 2.5 | DTT | 1 | 0 | 1 | 2 |
| 5 | PEGNB_{CA} | 4 | CGPQG* | 0.9 | 1 | 1 | 2 |
| (hiPSC) | | | IWGQC | | | | |
| 5 (DPSC, | PEGNB_{CA} | 4 | CGPQG* | 0.936 | 1 | 1 | 2 |
| PANC-1) | | | IWGQC | | | | |
| 6 | PEGNB-X | 4 | DTT | 1 | 0 | 1 | 2 |
| 9 (A), (B) | PEGNB-D | 4 | DTT or | 1 | 0 | 1 | 2 |
| | | | PEG4SH | | | | |
| 9 | PEGNB_{CA} | 4 | DTT | 1 | 0 | 1 | 2 |
| | +PEGNB-D | (in total) | | | | | |
| 11 | PEGNB-X | 4 | DTT | 1 | 0 | 1 | 2 |
| 10 | PEGNB_{CA} | 10 | DTT | 1 | 0 | 5 | 5 |
| | +PEGNB-D | (in total) | | | | | |
| 12, 13 | PEGNB_{CA} | 4 | CGPQG | 0.936 | 1 | 1 | 2 |
| | +PEGNB-D | | IWGQC | | | | |

For gravimetric swelling measurements, hydrogels were fabricated similarly to those for rheological testing except that the precursor solutions were prepared in double-distilled water (ddH2O)). See formulation details in Table 5. The fabricated hydrogels were vacuum dried to obtain dried gel weight (W_{dry}) prior to incubated in PBS at 37° C. or room temperature. The swollen gel weight (W_{swollen}) was recorded at predetermined time point. The mass swelling ratio (Q) is then defined as Q=Ws_{swollen}/W_{dry}. All results were averaged from 3 replicates.

Pseudo-First Order Hydrolytie Degradation Model Fitting

The remaining ester fraction in PEONBca and PEGNB-X hydrogels can be described by G′(t)/G′₀ in addition to the swelling ratio. For a pure ester hydrolysis system, the degradation kinetics can be expressed by a pseudo-first order equation.^{1,3,4} Thus, the apparent hydrolysis rate constant (k_{hyd}) may be obtained from G′(t)/G′₀∝e^{-k}^{_{hyd}}^{t} or q/q₀∝e^{k}_{hyd}^{t} or q/q₀∝e^{k}^{_{hyd}}^{t}.

Cell Culture and the Subsequent Encapsulation in PEGNBcs Hydrogels

Cellartis hiPSC line 12 (hiPSC) were maintained in Essential 8 (E8) medium (Gibco) on tissue culture plates coated with vitronectin (Gibco) per manufacturer's protocol. Media was refreshed daily, and passaging was conducted using TrypLE Select reagent (Gibco) treatment for 5 minutes every 3-4 days. During the first 24 hours after passaging, media was supplemented with ROCK inhibitor Y-27632 (10 μM) to prevent cell death. For encapsulated iPSCs, Y-27632 (10 μM) was added to E8 medium for the duration of the experiment. Dental pulp stem cells (DPSC) were cultured in low glucose Dulbecco's modified Eagle's medium (DEME, Gibco) containing 10% fetal bovine serum (FBS, Gibco) and 1% penicillin-streptomycin (Gibco) incubated at 37° C. with 5% CO₂. DPSC were trypsinated by 0.5% trypsin and suspended in hydrogel precursor solution at 2 Mcell/mL. PANC-1 cells, a human pancreatic cancer cell line, were cultured in high glucose Dulbecco's modified Eagle's medium (DEME, Gibco) containing 10% fetal bovine serum (FBS, Gibco) and 1% penicillin-streptomycin (Gibco) incubated at 37° C. with 5% CO₂. PANC-1 cells were trypsinated by 1% trypsin and suspended in hydrogel precursor solution at 2 Mcell/mL.

To encapsulate cells in hydrogels, 25 uL of cell precursor solution (see formulation details in Table 5) was loaded in a cut-opened I ml syringe and exposed to UV light (λ=365 nm, 5 mW/cm²) for 2 min. The cell-laden hydrogels were then cultured in regular cell culture media. The cell viability was evaluated by staining hydrogels with calcein AM/ethidium homodimer 1 live/dead staining kit prior to confocal imaging (Olympus Fluoview FV100 laser scanning microscope). Images were z-staged from 10 μm slices over 100 μm in total.

### Example 5

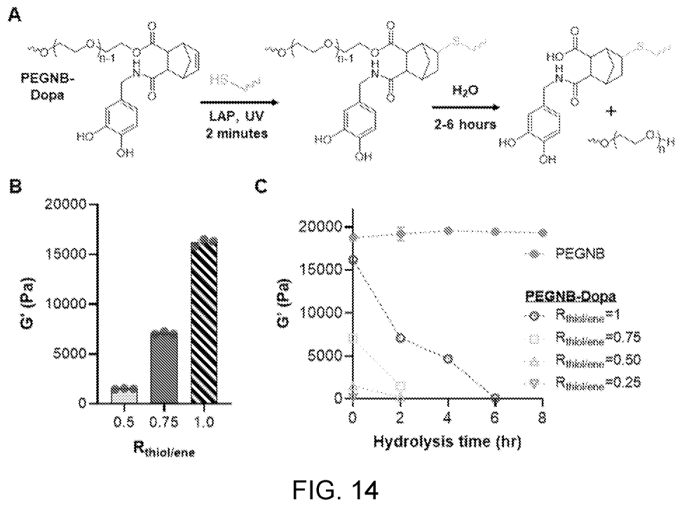

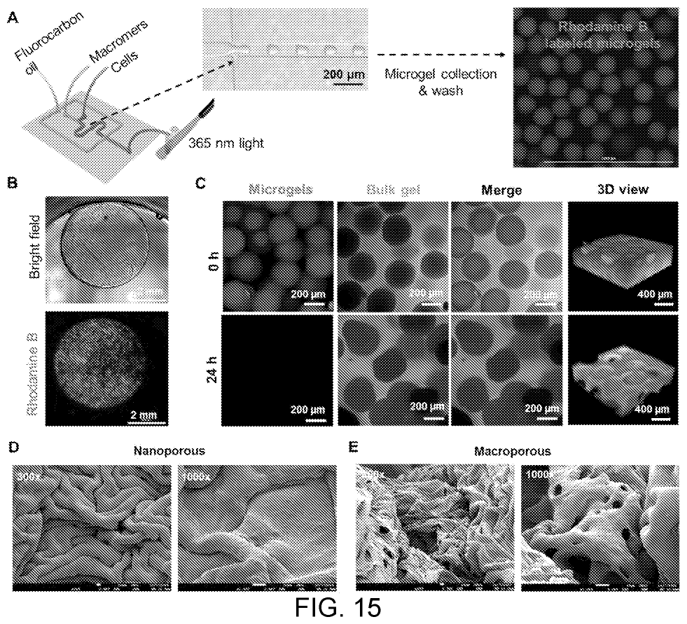

Degradation of PEGNB-Dopa hydrogels. PEGNB-Dopa, as prepared in Example 4, was chosen for forming dissolvable microgels. PEGNB-Dopa possesses the same rapid and orthogonal crosslinking efficiency afforded by the cytocompatible thiol-norbornene photopolymerization. Once crosslinked, PEGNB-Dopa based hydrogels would degrade rapidly in aqueous buffer at 37° C. via enhanced ester hydrolysis. FIG. 14 demonstrates the tunable crosslinking and rapid degradation of PEGNB-Dopa hydrogels. Panel A provides the chemical structure of PEGNB-Dopa along with the schematic of its gelation and hydrolytic degradation. Panel B provides initial shear moduli of PEGNB-Dopa hydrogels formed at varying thiol/ene ratio. Panel C shows the degradation of PEGNB and PEGNB-Dopa hydrogels formed with varying thiol/ene ratios as indicated by the decrease of shear modulus over time (n=3, Mean±SEM). Using PEG4SH as the crosslinker at different thiol/ene ratio, the photocrosslinking of PEGNB-Dopa into thiol-norbornene hydrogels was first evaluated. As with other thiol-norbornene hydrogels, the degree of hydrogel crosslinking scales with the thiol/ene ratio, with the highest shear moduli (G′) reaching 16 kPa at a stoichiometric ratio.

Regardless of initial crosslinking density (i.c., G′˜1.2 kPa to 16 kPa). all PEGNB-Dopa crosslinked hydrogels degraded rapidly in aqueous buffer, with gels crosslinked at low thiol/ene ratios degrading faster than at high thiol/ene ratios. For example, at a thiol/ene ratio of 0.5, PEGNB-Dopa/PEG4SH hydrogels degraded completely within 2 hours. In contrast, hydrogels crosslinked by conventional PEGNB did not exhibit any noticeable degradation within 8 hours. It is important to note that even bydrogels with high initial modulus (G′>16 kPa) could be completely degraded within 6 hours after incubating in water. The accelerated ester hydrolysis kinetics of PEGNB-Dopa hydrogels include a pseudo-first order degradation kinetic constant (kayi) of less than 1 hr-⁻¹. This is in stark contrast to the conventional hydrolytically degradable thiol-norbornene hydrogels that could only be degraded hydolytically over weeks or even months. Given that stiffer hydrogels can be easily handled and processed in a microfluidic droplet generator, PEGNB-Dopa bydrogels crosslinked at stoichiometric thiol/ene ratio were used for the following examples.

### Example 6

Characterization of macroporous hydrogels. The rapid degradation of PEGNB-Dopa hydrogels could be formulated into dissolvable microgels, serving as sacrificial templates for forming macroporous hydrogels. To achieve this, tandem droplet-microfluidics and thiol-norbornene photopolymerization were employed to synthesize near-monodispersed PEGNB-Dopa microgels. FIG. IS shows the fabrication and characterization of macroporous hydrogels. Panel A is a schematic of tandem microfluidic droplet generation and thiol-norbornene photopolymerization to form monodisperse microgels. Panel B provides bright field and fluorescence images of a microgel-templated hydrogel. Panel C provides confocal images of the microgel-templated hydrogel. The dissolution of the microgels resulted in a macroporous hydrogel. Panel D shows the dissolution of the microgels resulted in a macroporous hydrogel. Panel D provides SEM images of nanoporous hydrogels. Panel E provides SEM images of macroporous hydrogels. Scale bar=10 μm.

After the tandem droplet-microfluidics and thiol-norbornene photopolymerization, the PEGNB-Dopa microgels (crosslinked in the presence of thiolated Rhodamine B for visualization) were collected and further encapsulated in another bulk hydrogel. Of note, the bulk hydrogels were crosslinked by the same thiol-norbornene photopolymerization using conventional PEGNB, followed by incubating the composite gels in water to allow for dissolution of the encapsulated microgels. To facilitate the visualization of macro-scale pore formation. 0.1 wt % FITC-PEG-SH was added during the bulk hydrogel crosslinking. Confocal imaging revealed that the rhodamine-labeled microgels were dispersed in the FITC-labeled bulk gel. After 24 hours of incubation in PBS at 37ºC, minimal red fluorescence was detected, indicating the successful dissolution of the microgels. Compare FIG. 15, Panel C, at 0 hours and at 24 hours. As such, macroporous hydrogels were obtained without the use of any solvent or change in temperature for the removal of the microgel porogens. It can be seen from the confocal images that some microgels were in close contact, resulting in some level of interconnectivity of the pores.

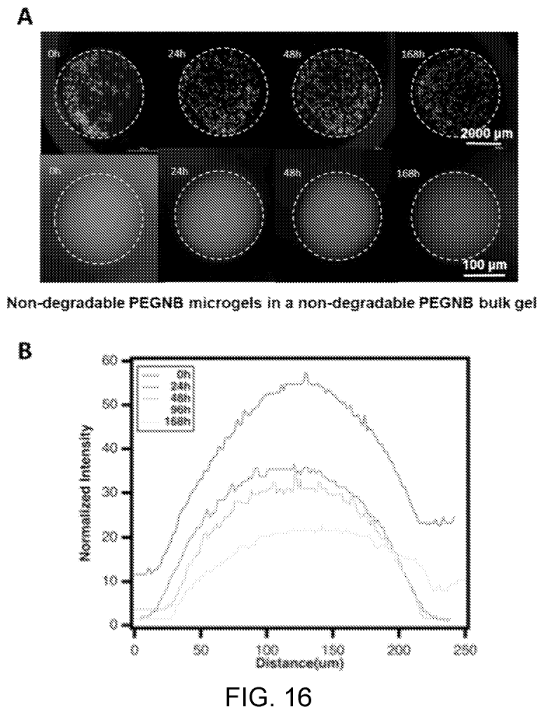

A control experiment using non-degradable PEGNB microgels resulted in minimum change of fluorescence even after 168 hours of incubation. FIG. 16 provides characterization data obtained in the control experiment. Panel A provides fluorescence images of non-degradable PEGNB microgels templated within PEGNB bulk hydrogels. Panel B provides Fluorescence intensity across a microgel over time.

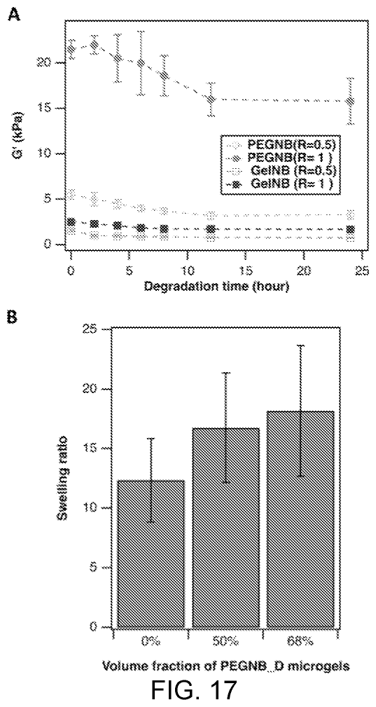

The formation of cavities with PEGNB-Dopa microgels was further confirmed by SEM imaging, where large pores were only observed in PEGNB-Dopa microgel templated bydrogels, as shown in Panels D and E of FIG. 15. Tracking moduli of the composite hydrogels revealed that the degradation process was completed within 24 hours, as stable moduli were achieved afterwards, as shown in FIG. 17. Panel A of FIG. 17 shows moduli changes of macroporous hydrogels. Bulk gels were crosslinked by PEGNB or GelNB with thiol/ene ratio (R) of 0.5 or 1. Panel B provides the swelling ratio of macroporous hydrogels crosslinked with PEGNB with varying volume fraction (0%, 50%, 68%) of PEGNB-Dopa microgels. Stiffness of the macroporous hydrogels was readily tuned depending on continuous matrix employed, in this case, PEGNB or gelatin-porbornene (GelNB) with 0.S or 1 thiol/ene ratio (FIG. 17, Panel A). Increasing the volume fraction of PEGNB-Dopa microgels within the continuous matrix (0%, 50%, or 68%) led to a slight increase in swelling ratio (FIG. 17, Panel B). indicating that the creation of the pores did not significantly alter the macroscopic hydrogel mechanical properties. Taken together, these results indicate success fabrication of macroporous hydrogels through using dissolvable PEGNB-Dopa microgels.

### Example 7

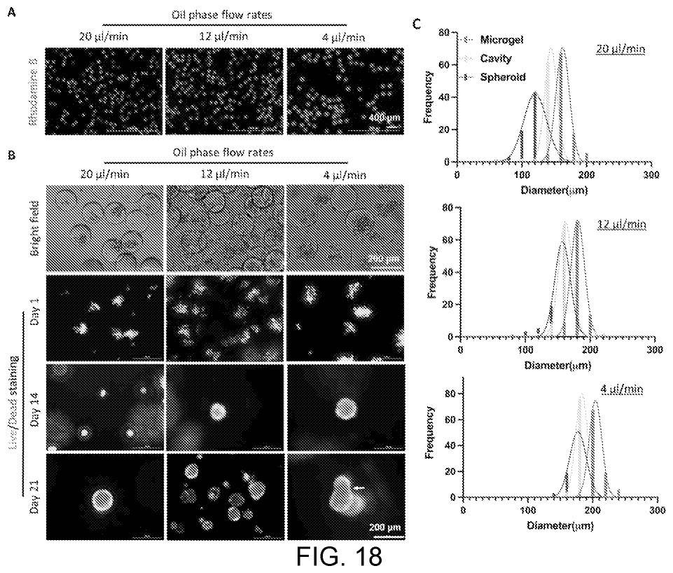

In situ formation of tumor spheorids in macroporous hydrogels. Droplet microfluidic devices have been used for in situ encapsulation of cells and were employed to encapsulate cells within the PEONB-Dopa microgels. An advantage of using droplet microfluidic device to fabricate microgels is that the size of the microgels can be precisely controlled by adjusting the flow rates of the oil and/or aqueous phases. For example, at a constant aqueous phase flow rate of 4 μl/min, decreasing oil phase flow rate from 20, 12, to 4 μl/min led to increasing diameter of the microgels (ca. 150 μm, 170 μm and 200 μm, respectively), FIG. 18 shows A549 spheroid formation within macroporous hydrogels. Microgels with different sizes were synthesized by controlling flow rate of the oil phase while keeping flow rate of the macromer phase at 4 μl/min, as shown in Panel A of FIG. 18. The cytocompatibility of this process was demonstrated by in situ encapsulation of A549 lung epithelial cells in the microgels, followed by a secondary encapsulation of the cell-laden microgels in a non-degradable, bio-inert PEGNB hydrogel. Live/dead staining results showed that A549 cells entrapped in the macroporous hydrogels cells remained alive and formed loosely packed cell clusters within the porous bulk gel day 1 post-encapsulation. Panel B provides bright field images (day 1) and Live/Dead staining of A549 cells on day 1, 14, and 21 post-encapsulation. Since non-degradable, bio-inert PEGNB was employed as the continuous matrix, the entrapped cells were not provided with an adhesive surface and were forced into spheroids at concave interfaces as microgels degraded. Over 21 days of culture, the cells assembled and grew into larger solid spheroids. When multiple microgels coalesced together, irregularly shaped cavities formed following the dissolution of the microgels. As a result, spheroids formed with irregular and elongated shape, as shown at Day 21 of Panel B of FIG. 18. Note that the spheroid size is dependent on the size of cavities, which is ultimately controlled by the size of the dissolvable microgels. Panel C of FIG. 18 provides quantification of the diameter of microgels, formed cavities, and spheroids as a function of oil phase flow rate. Increasing microgel diameters led to enlarged cavities and therefore, larger spheroids.

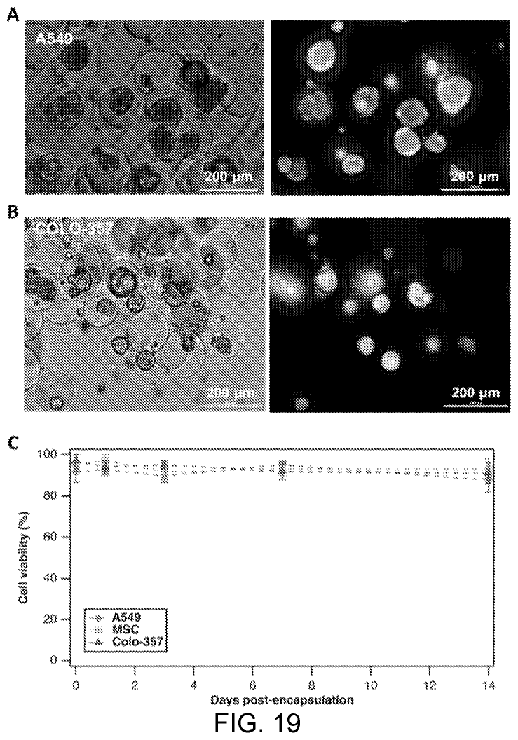

The process was compatible with other cell types, including a pancreatic cancer cell line, COLO-357. FIG. 19 provides further characterization of A549 cells and COLO-357 cells. Panel A provides live/dead staining of AS49 cell spheroids formed in macroporous hydrogels. Panel B provides live/dead staining of COLO-357 cell spheroids formed in macroporous hydrogels. Panel C shows cell viability after culturing for 14 days in macroporous hydrogels.

### Example 8

Assembly of solid spheroids from mMSCs using PEGNB as continuous matrix. Emerging evidence has shown that the secretome of MSC spheroids differs drastically from that of single cells. However, unlike cancer cells that prefer growing into clusters in 3D hydrogels. MSCs do not naturally grow into spheroids in chemically crosslinked gels, necessitating an engineering strategy for aggregating the cells. By encapsulating MSCs into macroporous hydrogels, it was believed that MSCs would undergo a self-assembly process similarly to that of the A549 cells. To prevent settlement of cells in the syringes during emulsification, the density of the aqueous phase containing cells was adjusted (with OPTIPREPTM, Sigma Aldrich) to match that of the cells (ca. 1.06 mg/ml). Failure to minimize fluid density differences would result in cells either settling down or aggregating in the syringe, reducing uniformity of the cell-to-droplet ratio. The ability to maintain cell viability while dispersing them within the carrying media for hours may be helpful for scaling up the production of cell-laden microgels. Unlike cancer cells that tend to grow as spheroids, the ultimate size of engineered MSC spheroids depends on the initial number of cells used instead of microgel size.

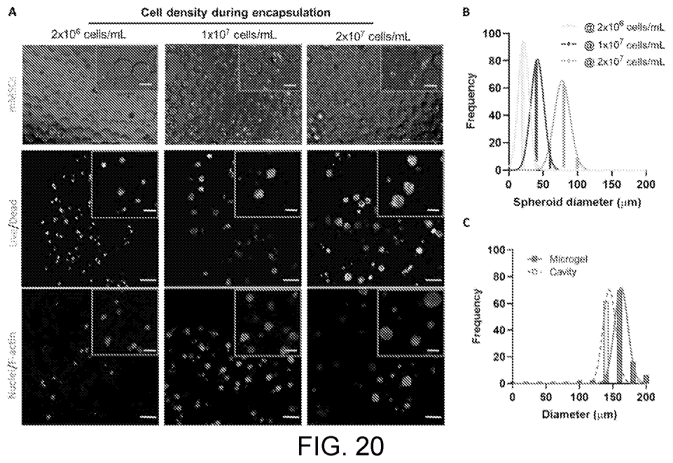

Mouse MSC (mMSC) was utilized to demonstrate in situ formation of MSC spheroids in the macroporous hydrogels. mMSC density was adjusted from 2×10⁶ cells/mL to 2×10⁷ cells/mL. The flow rates for cell stream, macromer stream, and oil stream were kept constant at 2 μl/min. 2 μl/min, and 20 μl/min, respectively. Above 90% of the encapsulated cells remained alive following the microfluidie encapsulation process. FIG. 20 provides characterization data for mMSC solid spheroid formation within macroporous hydrogels. Panel A provides live/dead staining of mMSCs immediately after microencapsulation into PEGNB-Dopa microgels. mMSCs with a density of 2×10⁶ cells/mL, 1×10⁷ cells/mL, and 2×10⁷ cells/mL in OPTIPREP™ were used for encapsulation. Panel B provides the spheroid diameter distribution profile obtained using the various densities. Panel C provides quantification of the sizes of microgels, cavities, and mMSC spheroids formed with various cell loading concentrations after 7 days of culture.

The microgel-encapsulated mMSCs formed spheroids over 7 days of culture within a continuous matrix crosslinked by non-degradable PEGNB and PEG4SH. The majority of the cells remained alive in the macroporous hydrogels, indicating the cytocompatibility of this platform to support the formation of solid and viable MSC spheroids, as shown in FIG. 20, Panel A. Cytoskeleton staining of F-actin and nuclei revealed that assembled MSC clusters have spherical morphology with limited spreading/invasion into the bulk matrix. Additionally, mMSC spheroids sizes were positively correlated to the cell density. Specifically, increasing cell concentration from 2×10⁶ cells/mL to 2×107 cells/mL led to MSC spheroids averaging 20 to 80 μm in diameter, as shown in FIG. 20, Panel B. Furthermore, the sizes of mMSC spheroids were all smaller than the sizes of the cavities/pores, which themselves were slightly smaller than the sizes of the microgels. See FIG. 20, Panel C. The smaller cavity/pore size was a result of bulk hydrogel swelling, whereas the much smaller spheroids may be attributed to the limited proliferation of mMSC spheroids.

To further enlarge the size of MSC spheroids, cells may be further concentrated within the carrying media. Alternatively, microgel size could be reduced to increase the cell-to-droplet ratio. One of the other benefits for using this platform to form macroporous hydrogels is that the mechanical or physiological properties of the continuous matrix can be easily customized to induce differential cell behaviors, such as cell attachment and differentiation. When thiol/ene ratio in continuous non-degradable PEGNB hydrogel was lowered to 0.5, overall stiffness of the hydrogels was decreased to 5 kPa, as shown in FIG. 17, Panel A. However, MSC spheroids were formed identically, in terms of size and shape, to those formed in the stiffer bulk hydrogels (data not shown), presumably due to limited cell-matrix interactions as the cavity sizes were considerably larger than the spheroid sizes. See FIG. 20, Panels B and C.

### Example 9

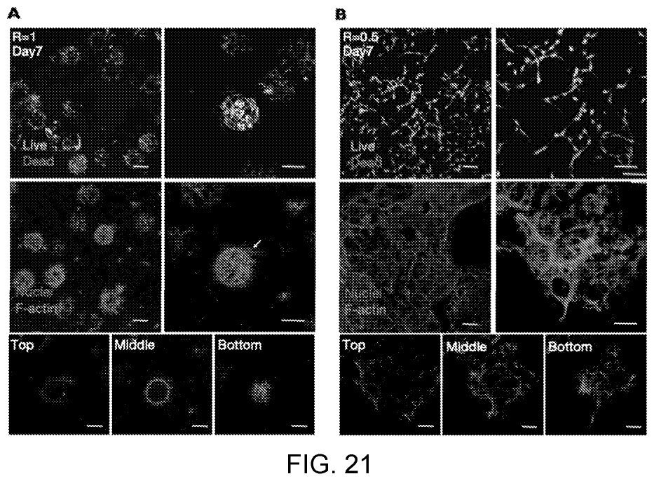

Assembly of hollow spheres from mMSCs using GelNB as continuous matrix. When a non-degradable inert PEONB hydrogel was used as the continuous matrix, there was limited cell-matrix interaction, which facilitated solid multicellular cluster formation, as shown in FIG. 20. When cell adhesive and protease-degradable GelNB was used to encapsulate MSC-laden microgels, cells were allowed to attach and degrade the bulk matrix following the dissolution of the PEGNB-Dopa microgels. Indeed, after 7 days of incubation, MSCs stayed alive and appeared attaching to the continuous GelNB matrix and formed hollow spheres containing a lumen. FIG. 21 shows the effect of bulk hydrogel properties on the formation of multicellular mMSC structures. Panel A, which is confocal imaging, shows hollow spheres formed in stiff macroporous GelNB hydrogels (R_{thiol/ene}=1). The arrow indicates invasion of cells into the stiff GeINB hydrogel.

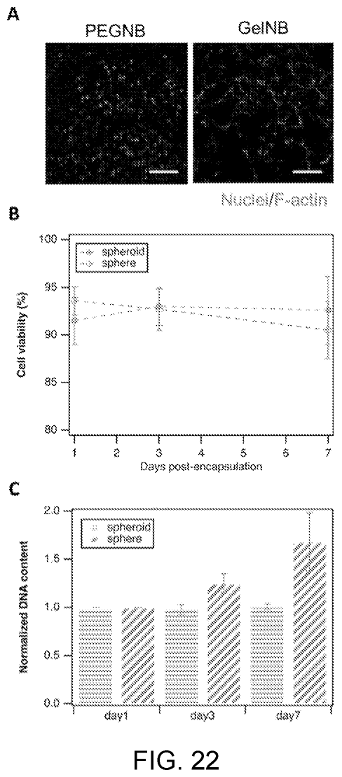

It was believed that cell migration was not an important factor in the initial phase of lumen formation owing to rapid degradation of the bioinert PEGNB-Dopa microgels, as shown in FIG. 14, Panel C. Instead, the encapsulated cells were “liberated” from the degrading microgels and simply ‘fell’ onto the surface of the continuous matrix. The “liberated” cells were allowed to adhere to the surface of the cell-adhesive GeINB hydrogel. Notably, cellular protrusion into the continuous matrix was observed, indicated by the arrow in FIG. 21, Panel A, indicating mMSCs can degrade their local network to a certain degree that allows cell penetration into the matrix. Interestingly, the ability of the encapsulated cells to form hollow cell spheres was affected by the shear modulus of the bulk hydrogels as lowering the bulk gel modulus (R=0.5) led to extensive cell spreading and the formation of an interconnected cellular network. FIG. 21, Panel B shows imaging of the interconnected cellular network formed in soft GelNB hydrogels (R_{thiol/ene}=0.5). Scale bar=100 μm. This was because the soft bulk GelNB hydrogels permitted MSCs to rapidly degrade and invade the surrounding matrix. In contrast, MSCs encapsulated in nanoporous PEGNB hydrogels exhibited a round morphology, while they had a more spreading morphology in GelNB hydrogels, as shown in Panel A of FIG. 22.

The results indicate that within macroporous hydrogels, the competition of cell-cell and cell-matrix interactions could be controlled by tuning bulk matrix adhesiveness and stiffness. It is worth noting that in this contribution only bioinert (PEGNB) and bioactive (GeINB) macromers were utilized for the crosslinking of bulk hydrogels. However, the unique ability to customize cell assembly structures using degradaing PEGNB-Dopa microgels could make this platform an enabling technique for other biomaterials (e.g., alginate or RGD-conjugated alginate), which may find extensive applications in engineering lumen-containing tissues in a geometrically defined environment.

### Example 10

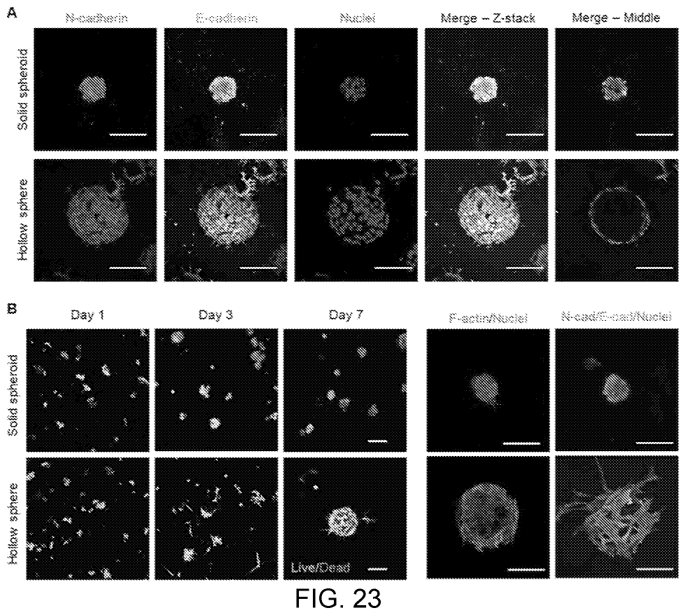

Evaluation of cell structures formed with mouse or human MSCs. To investigate whether cellular structures would affect cell phenotype, E-cadherin and N-cadherin were stained in MSC solid spheroids and hollow spheres. FIG. 23 provides fluorescence imaging of solid spheroids and hollow spheres formed with MSCs. Panel A shows immunofluorescence staining of E/N-cadherin and nuclei in mMSC solid spheroids or hollow spheres on day 7. Panel B provides Live/Dead staining and immunofluorescence staining of cytoskeleton, E/N-cadherin and nuclei in hMSC solid spheroids or hollow spheres. Scale bar=100 μm.

### Example 10

Both E- and N-cadherin were detected in MSC solid spheroids and hollow spheres (FIG. 23, Panel A), indicating these structured MSCs still preserved both epithelial and mesenchymal phenotypes. The simultaneous expression of both cadherins indicates the quiescent state of the structured MSCs, and they may be favorably induced into functional cell types following differentiation or transdifferentiation. A single confocal image at the middle section revealed distinct cellular structures with either solid or hollow interior. See FIG. 23, Panel A.

Human MSCs (hMSCs) were used to investigate whether this cell-assembly platform could be applied to human cells, which were typically larger than marine cells. FIG. 21 provides characterization data for these cells. Panel A provides cytoskeleton staining of singular mMSCs encapsulated within nanoporous PEGNB or GelNB bulk hydrogels. Scale bar=100 μm. Panel B shows viability of hMSC spheroids and spheres after encapsulation. Panel C shows normalized DNA content of hMSC spheroids and spheres. hMSCs survived the macroporous hydrogel forming process with a viability above 90% in both human and murine experiments (FIG. 22, Panel B) and self-assembled into either solid spheroids or hollow spheres, depending on the cell adhesiveness of the continuous matrix (FIG. 23, Panel B). It is worth noting that hMSCs did not proliferate when forming spheroids, while they can proliferate in the form of bollow spheres, as indicated by fluorescent images (FIG. 23, Panel B) and DNA quantification (FIG. 22, Panel C). Meanwhile, structured hMSCs also preserve epithelial and mesenchymal phenotypes, as indicated by the expression of E- and N-cadherin (FIG. 23, Panel B). Importantly, both cadherins have been related with MSC paracrine activity and fate decision, thus, the strong expression of E/N-cadherin in structured MSCs may affect their therapeutic potentials.

### Example 11

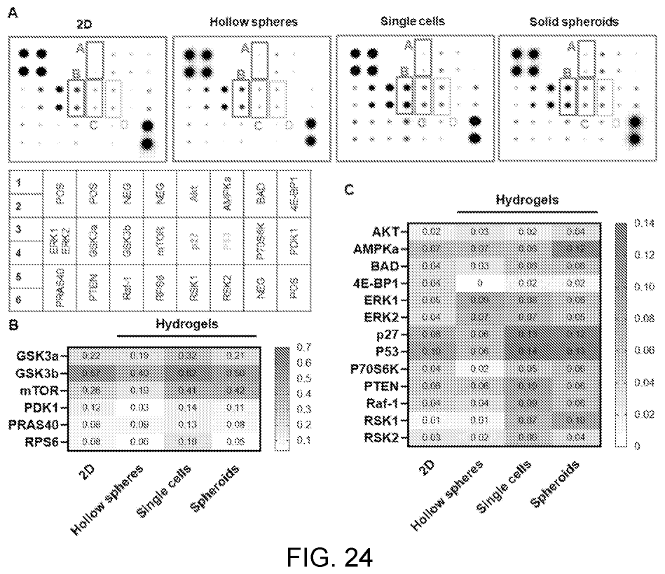

AKT pathway activation in bMSCs with different 3D growth pattern. MSCs can sense and response to the properties of the extracellular matrix via activating intracellular signaling pathways. Additionally, recent studies have discovered that MSC spheroids exert drastically different trophic factor secretion profiles due to heightened cell-cell interactions. Therefore, structured/assembled MSCs could be highly advantageous toward engineering MSC-based therapeutics. To this end, cell lysates were extracted from hMSCs cultured in spheroids and hollow spheres formed in macroporous hydrogels and phosphorylation levels of 19 proteins involved in the AKT signaling pathway were evaluated. FIG. 24 provides characterization data using the AKT pathway. Panel A provides arrays of blotted membranes using hMSC lysates from 2D TCPs, 3D encapsulated single cells, as well as hollow spheres or solid spheroids formed in macroporous hydrogels. Panels B and C provide heatmaps of phosphorylation level of AKT pathway for various proteins.

### Example 11

hMSCs cultured on 2D TCPs and encapsulated in 3D nanoporous hydrogels were used as controls, as shown in FIG. 24, Panel A. AKT pathway was studied here due to its diverse role in cell proliferation, survival, and apoptosis. The levels of protein phosphorylation were analyzed and grouped into two heat maps with high (FIG. 24, Panel B) and low (FIG. 24. Panel C) signals for easy visualization of the differences between proteins and culture conditions. Overall, the phosphorylation levels of the AKT pathway proteins in hMSC hollow spheres were similar to that in 2D culture, while the AKT protein phosphorylation levels in 3D single cells were similar to that in spheroids. Another phenomenon worth noting was the high level of phosphorylation of many AKT pathway proteins in 3D hMSC cultures, both in solid spheroids and direct encapsulation of dispersed single cells. While endogenous (Le., without using an activator) phosphorylation of AKT was similar among the four groups (FIG. 24, Panel B), downstream proteins that are positively regulated by AKT phosphorylation, including mTOR, P70S6K, and RPS6, had higher phosphorylation in single cells and solid spheroids than in 2D and hollow spheres. In addition, the phosphorylation of PDKI, a growth-promoting protein upstream of AKT activation, was similar between solid spheroids and single cells, but substantially lower in hollow spheres. On the other band, the phosphorylation of PTEN, a growth-inhibiting protein upstream of AKT, was highest in the single cells group, indicating that the lack of cell-cell interaction is unfavorable for proliferation of hMSCs in 3D. Another notable difference was the higher phosphorylation of p27 and PS3 in single cells and solid spheroids. p27 inhibits cell cycle progression, while P53 is a known growth suppressor. The higher phosphorylation of these two proteins in 3D single bMSCs and spheroids suggested that the cells in these formats may experience lower proliferation capacity.

These results suggest that both cell-materials and cell-cell interactions affect the autonomous activation of AKT signaling. Of note, both hMSCs cultured in 2D TCP and in hollow spheres in macroporous hydrogels formed monolayer-like structures with polarized cell-cell interactions (i.e., an apical surface facing media/cavity and a basal surface adhering to TCP/hydrogel). On the other hand, hMSCs encapsulated as single cells or assembled in spheroids did not exhibit substantial epical/basal polarization (FIG. 23). The apical/basal polarity is implicated in stem cell fate processes.

### Example 12

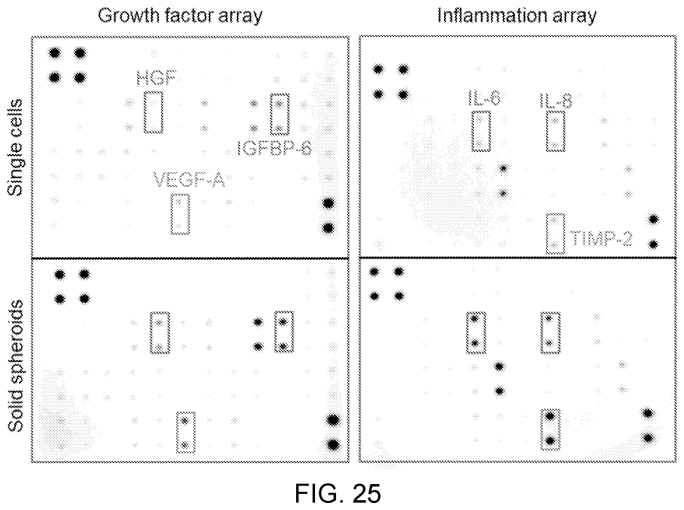

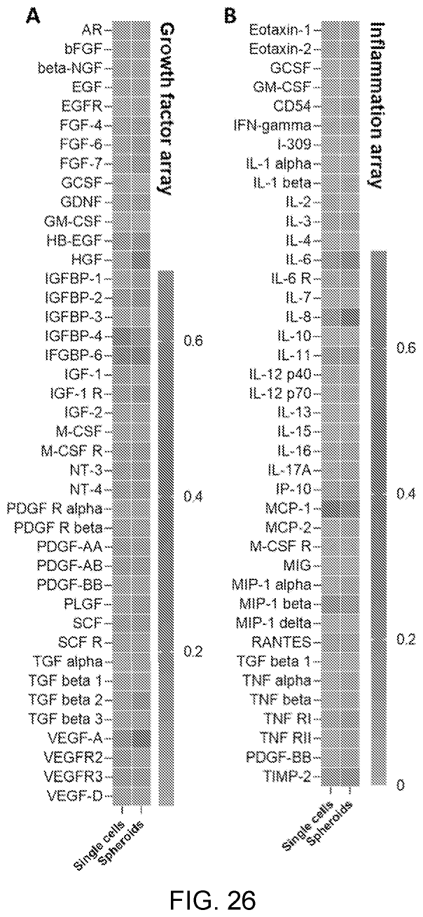

Secretory properties of assembeled hMSCs. To further evaluate whether assembly of hMSCs into solid spheroids would improve secretory properties, conditioned media (CM) was collected from bMSCs encapsulated as single cells or that formed solid spheroids in the macroporous hydrogels. These two conditions were selected for comparison for their similarity in AKT pathway activation (FIG. 24). CM were subjected to antibody array for detecting arrays of growth factors and inflammatory cytokines (FIG. 25). FIG. 26 provides heatmaps for growth factors and inflammation cytokines secreted in the CM produced from hMSCs encapsulated as single cells or assembled as spheroids in the macroporous hydrogels. CM were collected from three hydrogel samples per condition. Panel A shows growth factor secretion. The secretion of several growth factors and receptors, including FGF-6, FOF-7, HOF, IGFBP-1, IGFBP-4, IGFBP-6. IGF-IR, PLGP, SCF. TOFB2, and VEGF-A was noticeably enhanced in hMSC spheroids relative to direct 3D cell encapsulation. Panel B shows inflammation cytokine secretion. hMSCs also secreted higher amount of IL-6, IL-8, and TIMP-2 once they were assembled into spheroids.

IL-6 and IL-8 are involved in regulating inflammatory response, reaffirming that hMSC spheroids can be leveraged to regulate regeneration via their secretomes. On the other hand, the increased secretion of TIMP-2, which inhibited activity of matrix metalloproteinase 2 (MMP2). suggested that the assembled hMSC spheroids exhibited reduced migratory potential due to a reduced level of MMP activity. While this panel of growth factor and inflammation cytokine arrays only represent a fraction of MSC secretomes, and without intending to be bound by any particular theory, it is believed that the hMSC spheroids secretomes could be beneficial for regenerative medicine applications.

The present example explored MSC assembly and secretome profiles using the macroporous hydrogels templated by dissolvable PEGNB-Dopa microgels. Given that the PEGNB-Dopa can be readily crosslinked into hydrogels with any shapes or sizes through the efficient thiol-norbornene photopolymerization, this material platform may have high potential for creating sophisticated internal structures via biofabrication techniques. For example, the dissolvable PEGNB-Dopa microgels can be encapsulated in sufficiently high density to create an interconnected porous structure. PEONB-Dopa may also be photocrosslinked into other shapes (e.g., fibers, cylindrical posts, etc.) for serving as a rapidly dissolving sacrificial material compatible with in situ cell encapsulation. These hierarchical hydrogel structures may be used, for example, for basic scientific research or to facilitate tissue regeneration via enhancing cell infiltration or vascularization.

In conclusion, a biomaterial platform for rapid in situ formation of cell-laden macroporous hydrogels was developed. Central to this platform was the use of PEGNB-Dopa microgels as the sacrificial porogens, which rapidly dissolved upon contacting with an aqueous solution. As the dissolution of the microgels did not rely on any external triggers or changes in environmental conditions (e.g., pH, temperature), this in situ pore-forming approach was high cytocompatible, as demonstrated by the in situ encapsulation of multiple cell types. Furthermore, adjusting the cell adhesiveness of the bulk hydrogels afforded the formation of solid cell spheroids or hollow spheres. The assembly of hMSC spheroids/spheres led to differential activation of the AKT pathway. with the solid spheroids exhibiting robust secretion of growth factors and certain cytokines. In summary, this platform provides an innovative method for forming cell-laden macroporous hydrogels for a variety of biomedical applications.

### Example 13

### Materials and Methods

Materials, PEG-OH (8-arm, 20 kDa, JenKem), carbic anhydride (Sigma-Aldrich), 5-norborbene-2-carboxylic acid (Sigma-Aldrich), 4-dimethylaminopyridine (DMAP, Alfa Aesar), dopamine hydrochloride (Sigma-Aldrich), tyramine hydrochloride (Chem-Impex), isopropylamine (TCI chemicals), N,N′-Diisopropylcarbodiimide (DIC, Chem-Impex), N,N′-Dicyclohexylcarbodiimide (DCC, Sigma-Aldrich), hydroxybenzotriazole (HOBI, ≥20 wt % water, Oakwood Chemical), N,N′-diisopropylethylamine (DIPEA, TCI chemicals), cysteine (Sigma-Aldrich), and lithium phenyl-2,4,6-trimethylbenzoylphosphinate (LAP, >95%, Sigma-Aldrich), 4-arms thiolated PEG (PEG4SH, 10 kDa, Laysan Bio), 5,S′-dithiobis-(2-nitrobenzoic acid) (Ellman's reagent, Life Technologies), pyridine (Fisher), anhydrous tetrahydrofuran (THF, Fisher), anhydrous dimethylformamide (DMF, amine-free. Fisher), anhydrous dichloromethane (DCM, Fisher), methanol (Sigma-Aldrich) and diethyl ether (Fisher) were used as received.

Synthesis of fonctionalized macromers, PEGNB was synthesized according to previously established protocols. Before conducting the reaction, PEG was distilled to remove excess water. To activate 5-norbornene-2-carboxylic acid (norbornene acid hereafter), 1.22 mL. norbornene acid (10 mmol, 5 equiv.) was combined with 1.03 g DCC (5 mmol, 2.5 equiv.) in 30 mL anhydrous DCM under N₂ for 1 h. Meanwhile, 5 g dry PEG-OH (hydroxy group 2 mmol), 122 mg DMAP (1 mmol, 0.5 equiv.), 810 AL pyridine (10 mmol, 5 equiv.) and 30 mL anhydrous DCM were charged in a separate round bottom flask. After I h, the white precipitate generated during the activation step was filtered out. The activated norbornene acid solution was then drop-added into PEG solution at 0° C. under Na in dark. The reaction was allowed to proceed overnight. To ensure high conjugation efficiency, another portion of activated norbornene acid was drop-added into the reaction the next day. After the reaction was completed, the solution was filtered and precipitated 2 times into diethyl ether. The crude product was vacuum dried and redissolved in pure water. The final product was harvested by dialyzing in pH 6 water for 3 days followed by lyophilization. The product was characterized by H NMR spectra.

PEGNBc; and PEGNB-Dopa were synthesized in accordance with Example 4. Briefly, 10 g PEG-OH (hydroxyl group 0.4 mmol), 3.28 g carbie anhydride (2 mmol, 5 equiv.). 0.49 g DMAP (0.4 mmol, 1 equiv.), and 83 ml. anhydrous THE were charged in a round-bottom flask equipped with a stir bar. The reaction was conducted at 60° C. After 12 h, another portion of carbic anhydride and DMAP were added into the reaction. The reaction was continued for another 24 h. PEONBCA was retrieved via precipitation through diethyl ether twice and subsequently dried in vacuum. To synthesize PEGNB-Dopa, 1 g PEGNBea (carboxylic group 380 μmol), 148.8 uL DIC (950 μmol, 2.5 equiv.), 160 mg HOBt (950 μmol, 2.5 equiv.) and 10 ml. amine-free DMF were charged into a reaction vial equipped with a stir bar. The activation of the acid group was blanketed under nitrogen gas for 2 h in dark. 180 mg dopamine hydrochloride (950 μmol, 2.5 equiv.). 165.4 AL DIPEA (950 μmol, 2.5 equiv.), and 23 mg DMAP (190 μmol, 0.5 equiv.) were dissolved in 1 ml amine-free DMF and then added into the reaction. The reaction was continued overnight. The product was dialyzed in methanol for one day and dried out in vacuum. The product was characterized by ¹H NMR spectra (Bruker 500 MHz).

GelNB was synthesized according to a previously described method. Briefly, 2 g of type B gelatin was dissolved in PBS to make a 10 wt % gelatin solution. 0.6 g of carbic anhydride was added to the gelatin solution, and pH was adjusted to 7.5-8. The reaction was allowed to proceed for 24 b, after which the product was dialyzed against DI water for 3 days and lyophilized.

Microfluidic chip fabrication, Microfluidic devices were designed and fabricated via conventional soft lithography as previously described. Briefly, polydimethylsiloxane (PDMS) was poured onto a silicon wafer (Wafer World Ine, USA) with micro-structures designed in CAD, vacuumed fot 60 minutes to remove entrapped air, then transferred to a 70° C.oven to cure overnight, PDMS replicas were then trimmed and punched with a 20 G dispensing needle (CML Supply, USA) to fashion inlets and outlets. After cleaning, PDMS replicas were bonded to glass slides after exposing to oxygen plasma (Harrick Scientific, USA), then transferred to 70° ° C.oven to facilitate bonding. Channel dimensions (h×w) were 100 μm×100 μm for cell and macromer stream, and 100 μm/20 μm for the oil stream.

Microgel fabrication, 10 wt % PEGNB or PEGNB-Dopa, 20 mM PEG4SH, 7.2 mM LAP, with 0.1 wt % thiolated Rhodamine B was prepared as hydrogel-forming solution, PBS or cell-containing media was mixed with hydrogel-forming solution on-chip via a serpentine channel. Microgels with a final concentration of 5 wt % PEGNB or PEGNB-Dopa. 10 mM PEG4SH and 3.6 mM LAP were fabricated under constant flow rates for both PBS and macromer solution (2 μL/min) while varying oil phase flow rate to 4 μL/min, 12 μL/min, and 20 pL/min to create microgels of different sizes. Droplets were then exposed to 15 mW/cm² UV for 20 seconds for gelation. Polymerized microgels were separated from oil and recovered into PBS by centrifugation on a 40 pun cell strainer.

Macroporous or nanoporous hydrogel fabrication and characterization. After separating from oil, microgels were immediately encapsulated within a continuous hydrogel matrix to form macroporous hydrogels. Either PEGNB (5 wt %, 5 (R=0.5) or 10 mM (R=1) PEGASH, 3.6 mM LAP), or GelNB (5 wt %, 0.5 (R=0.S) or 1 mM (R=1) PEG4SH, 3.6 mM LAP) was used to form continuous matrix. and 30 μl solution was exposed to 15 m W/cm² UV light for 20 seconds in a 1 mL syringe with tip removed for gelation. Fluorescence images of the microgels were taken every 24 hours by using a fluorescent microscopy (Lionheart FX, BioTek) and fluorescence intensity was quantified in IMAGEJ™. Confocal microscopy was also utilized to verify the macroporous structure. For confocal imaging, both microgels and bulk gels were labeled with fluorescent dye (0.1 wt % thiolated-Rhodamine for microgels and 0.1 wt % FITC-PEG-SH for bulk gels). For measuring mechanical moduli, 45 μl hydrogel forming solution with/without microgels was injected in between two glass slides separated by 1 mm thick spacers. Hydrogels were swollen for 2 hours prior to measuring stiffness, which was done by using a digital rheometer (Bohlin CVO100, Malvern Instruments), with an 8 mm parallel plate geometry with a gap of 720 um. For measuring swelling ratio, polymerized hydrogels were incubated in PBS for 2 hours before measuring wet weight. Dry weight of the hydrogels was measured after lyophilizing for 48 hours. Swelling ratio was calculated as the ratio between hydrogel wet weight and dry weight.

Scanning electron microscopy (SEM) imaging. Non-degradable PEGNB bulk hydrogels and macroporous hydrogels were frozen in liquid nitrogen before lyophilization for 24 hours. And then the samples were imaged with a SEM after coating the hydrogel surface with gold.

Cell culture, microencapsulation and encapsulation within macroporous hydrogels. Mouse MSCs were a generous gift from Dr. Hiroki Yokota (IUPUI BMB). Human MSCs were isolated from deidentified bone marrow aspirate (Lonza), Mouse or human MSCs, lung epithelial cell line A549, pancreatic cancer cell lines Colo-357, and PANC-1 were cultured at 37° C. and 5% CO₂ in Dulbecco's Modified Eagle's Medium (DMEM, HyClone) with low glucose (high glucose for Colo-357 and PANC-1) supplemented with 10% fetal bovine serum (FBS, Corning, USA), 1% Antibiotic-Antimycotic (Life Technologies, USA). 2 ng/mL. FGF-2 was used when culturing MSCs. Cell populations were subcultured every 3 days. To prepare cells for microencapsulation, cells were trypsinized, pelleted, and resuspended to a final concentration of 2×10⁶, 2×10⁷ and 4×10⁷ cells/mL in culture media or OpuPrep for microencapsulation as described above, except that cell stream was separated from macromer stream for homogeneous cell encapsulation. Flow rates for cell stream and macromer stream were kept at 2 μl/min, while the flow rate of oil stream was varied to control the diameter of microgels. The concentration of macromer solution was doubled to 10 wt % PEONB-Dopa, 20 mM PEG4SH and 7.2 mM LAP, followed by encapsulation into macroporous hydrogels, where 30 μl solution was exposed to 15 m W/cm² UV light for 20 seconds in a 1 ml syringe with tip cut off for gelation. Cell-laden macroporous hydrogels were then transferred and cultured in the incubator. To encapsulate MSCs into PEGNB or GeINB nanoporous hydrogels, 500,000 cells/ml were suspended into hydrogel-forming solutions for forming 5 wt % PEGNB or GelNB hydrogels with PEGASH (R=0.5 or 1) as described above. 30 μl solution was exposed to 15 mW/cm² UV light for 20 seconds in a 1 ml syringe with tip removed for gelation.

Cell viability assay. Viability of encapsulated cells was measured by staining with Live/Dead viability assays (Biotium, USA), a cellular membrane integrity assay that stains live cells with green fluorescence and dead cells with red fluorescence. Cell viability was imaged using a fluorescence microscope and calculated based on three images.

Immunofluorescence staining. Encapsulated cells were fixed in 4% paraformaldehyde for one hour at room temperature on an orbital shaker, followed by washing with PBS three times. Then, cell-laden hydrogels were permeabilized in 0.5% Triton X-100 at room temperature for 10 minutes. The hydrogels were then washed with PBS and stained with 100 nM Rhodamine Phalloidin (Cytoskeleton Ine) and DAPI (1:1000) for 3 hours at room temperature. After the final wash, the z-stack images were obtained with confocal microscopy. To stain E and N-cadherin, hydrogels were fixed, blocked (5% BSA for 2 hours), permeabilized as described above, then incubated with mouse anti-E-cadherin (1:100) and rabbit anti-N-cadherin (1:100) primary antibodies in antibody diluent buffer at 4° ° C.overnight. After washing with 3% BSA for 30 minutes at 4° C., secondary antibodies Alexa 488 goat anti-mouse and Texas Red mouse anti-rabbit at a concentration of 10 ng/mL. were applied overnight at 4° C.to visualize the presence of primary antibodies. After washing with PBS, hydrogels were imaged with confocal microscopy.

AKT phosphorylation array. The AKT pathway phosphorylation array (RayBiotech US) was used to identify the phosphorylation level of proteins involved in the AKT signaling pathway, among structured hMSCs and bMSCs grown on 2D tissue culture plastics (TCPs) or 3D encapsulated as single cells in bulk PEGNB hydrogels. hMSCs were cultured for 7 days before lysis for the analysis of the AKT phosphorylation array. Cell lysate was detected with biotinylated detection antibodies and visualized via chemiluminescence. The intensity of the individual dot represented the amount of specific proteins and was normalized to the reference dots for quantification.

Growth factor and inflammation arrays, Secretion of cytokines and inflammatory factors was measured from CM of hMSC spheroids and single cells encapsulated in bulk hydrogels. hMSCs were cultured in microporous or nanoporous hydrogels for 3 days before CM was collected. CM was detected with biotinylated detection antibodies and visualized via chemiluminescence. The intensity of the individual dot represented the amount of specific proteins and was normalized to the reference dots for quantification.

Statistical analysis, All experiments were conducted independently for three times and results were reported as Mean±SD. One-way ANOVA and unpaired t-test with Welch's correction were used on PRISMIM software to analyze the statistical significance of the data. *, **, ***, **** represent p<0.05, 0.01, 0.001, 0.0001, respectively.

### REFERENCES

Each of the following documents is hereby expressly incorporated by reference herein in its entirety.

Levy O. Koai R, Siren E M, Bhere D, Milton Y, Nissar N, et al. Shattering barriers toward clinically meaningful MSC therapies. Science Advances. 2020;6:eaba6884.

Pittenger M F, Mackay A M, Beck S C, Jaiswal R K, Douglas R, Mosca J D, et al. Multilineage potential of adult human mesenchymal stem cells. science. 1999;284:143-7.

Guo X, Bai Y, Zhang L. Zhang B, Zagidullin N, Carvalho K, et al. Cardiomyocyte differentiation of mesenchymal stem cells from bone marrow: new regulators and its implications. Stem cell research & therapy. 2018;9:1-12.

Urrutia D N, Caviedes P, Mardones R, Minguell J J, Vega-Letter A M, Jofre CM. Comparative study of the neural differentiation capacity of mesenchymal stromal cells from different tissue sources: An approach for their use in neural regeneration therapies. PloS one. 2019;14:e0213032.

Katikireddy K R, Dana R, Jurkunas U V. Differentiation potential of limbal fibroblasts and bone marrow mesenchymal stem cells to corneal epithelial cells. Stem Cells. 2014:32:717-29.

Madrigal M. Rao K S, Riordan N H. A review of therapeutic effects of mesenchymal stem cell secretions and induction of secretory modification by different culture methods. Journal of translational medicine. 2014; 12:1-14,

Ranganath S H, Levy O, Inamdar M S, Karp J M. Harnessing the mesenchymal stem cell secretome for the treatment of cardiovascular disease. Cell stem cell. 2012; 10:244-58.

Yang H, Nguyen K T, Leong D T. Tan N S, Tay C Y. Soft Material Approach to Induce Oxidative Stress in Mesenchymal Stem Cells for Functional Tissue Repair. ACS applied materials & interfaces. 2016;8:26591-9.

Liu F D, Pishesha N. Poon Z, Kaushik T, Van Vliet K J. Material Viscoelastic Properties Modulate the Mesenchymal Stem Cell Secretome for Applications in Hematopoietic Recovery. 2017;3:3292-306.

Yang H, Cheam N M J, Cao H, Lee M K H, Sze S K, Tan N S. et al. Materials Stiffness-Dependent Redox Metabolic Reprogramming of Mesenchymal Stem Cells for Secretome-Based Therapeutic Angiogenesis. ACS applied materials & interfaces. 2019;8:e1900929.

Liu K, Veenendaal T, Wiendels M, Ruiz-Zapata A M, van Laar J, Kyranas R, et al. Synthetic Extracellular Matrices as a Toolbox to Tune Stem Cell Secretome. ACS biomaterials science & engineering. 2020;12:56723-30.

Caldwell A S, Rao V V, Golden A C, Anseth K S. Porous bio-click microgel scaffolds control hMSC interactions and promote their secretory properties. Biomaterials. 2020;232:119725.

Qazi T H, Mooney D J, Duda G N, Geissler S. Niche-mimicking interactions in peptide-functionalized 3D hydrogels amplify mesenchymal stromal cell paracrine effects. Biomaterials. 2020;230:119639.

Andrzejewska A, Dabrowska S, Lukomska B, Janowski M. Mesenchymal stem cells for neurological disorders. Advanced Science, 2021;8:2002944.

Gomez-Salazar M. Gonzalez-Galofre Z N, Casamitjana J. Crisan M, James A W, Péault B. Five decades later, are mesenchymal stem cells still relevant? Frontiers in bioengineering and biotechnology. 2020;8:148.

Wechsler M E, Rao V V, Borelli A N, Anseth K S. Engineering the MSC Secretome: A Hydrogel Focused Approach. Advanced healthcare materials. 2021;10:2001948.

Cipriano M. Freyer N. Knöspel F, Oliveira N G, Barcia R. Cruz P E, et al. Self-assembled 3D spheroids and hollow-fibre bioreactors improve MSC-derived hepatocyte-like cell maturation in vitro. Archives of toxicology. 2017;91:1815-32.

Whitehead J, Griffin K H, Gionet-Gonzales M. Vorwald C E, Cinque S B, Leach J K. Hydrogel mechanics are a key driver of bone formation by mesenchymal stromal cell spheroids. Biomaterials. 2021;269:120607.

He J, Zhang N. Zhu Y, Jin R, Wu F. MSC spheroids-loaded collagen hydrogels simultaneously promote neuronal differentiation and suppress inflammatory reaction through PI3K-Akt signaling pathway. Biomaterials. 2021;265:120448.

Sun X, Li K, Zha R, Liu S, Fan Y, Wu D, et al. Preventing tumor progression to the bone by induced tumor-suppressing MSCs. Theranostics. 2021;11:5143.

Kim T-H, Choi J H, Jun Y, Lim S M, Park S, Paek J-Y, et al. 3D-cultured human placenta-derived mesenchymal stem cell spheroids enhance ovary function by inducing folliculogenesis. Scientific reports. 2018;8:1-11.

Khetan S. Guvendiren M, Legant W R. Cohen D M, Chen C S, Burdick J A. Degradation-mediated cellular traction directs stem cell fate in covalently crosslinked three-dimensional hydrogels. Nature materials. 2013;12:458-65.

Chaudhuri O, Gu L. Klumpers D, Darnell M, Bencherif S A, Weaver J C, et al. Hydrogels with tunable stress relaxation regulate stem cell fate and activity. Nature materials. 2016:15:326-34.

Lin C-C, Metters A T. Hydrogels in controlled release formulations: network design and mathematical modeling. Advanced drug delivery reviews. 2006;58:1379-408.

Lin C-C, Anseth K S. PEG hydrogels for the controlled release of biomolecules in regenerative medicine. Pharmaceutical research. 2009;26:631-43.

Baraniak P, McDevitt T. Scaffold-free culture of mesenchymal stem cell spheroids in suspension preserves multilineage potential. Cell and tissue rescarch. 2012;347:701-11.

Lewis K J. Tibbitt M W, Zhao Y, Branchfield K. Sun X, Balasubramaniam V, et al. In vitro model alveoli from photodegradable microsphere templates. Biomaterials science. 2015;3:821-32.

Zhang Y S, Zhu C, Xia Y. Inverse opal scaffolds and their biomedical applications. Advanced Materials. 2017;29:1701115.

De France K J, Xu F, Hoare T. Structured macroporous hydrogels: Progress, challenges, and opportunities. Advanced healthcare materials. 2018;7:1700927.

Wang E, Wang D, Geng A, Seo R, Gong X. Growth of hollow cell spheroids in microbead templated chambers. Biomaterials. 2017;143:57-64.

Jiang Z, Shaba R, Jiang K, McBride R, Frick C, Oakey J. Composite hydrogels with controlled degradation in 3D printed scaffolds. IEEE transactions on nanobioscience. 2019;18:261-4.

Shaha R K, Jiang Z, Frick C P, Oakey J. Cell-laden particulate-composite bydrogels with tunable mechanical properties constructed with gradient-interface hydrogel particles. ACS Applied Polymer Materials. 2019;1:2571-6.

Loh Q L, Choong C. Three-dimensional scaffolds for tissue engineering applications: role of porosity and pore size. 2013.

Reissis Y, García-Gareta E, Korda M, Blunn G W, Hua J. The effect of temperature on the viability of human mesenchymal stem cells. Stem cell research & therapy. 2013;4:1-11.

Li Z, Fan Z, Xu Y, Lo W, Wang X, Niu H, et al. pH-sensitive and thermosensitive hydrogels as stem-cell carriers for cardiac therapy. ACS applied materials & interfaces. 2016;8:10752-60.

Griffin D R, Weaver W M, Scumpia P O, Di Carlo D, Segura T. Accelerated wound healing by injectable microporous gel scaffolds assembled from annealed building blocks. Nature materials. 2015;14:737-44.

Imaninezhad M, Hill L, Kolar G, Vogt K, Zustiak S P. Templated macroporous polyethylene glycol hydrogels for spheroid and aggregate cell culture, Bioconjugate chemistry. 2018;30:34-46.

Lin P-Y, Lin C-C. Facile Synthesis of Rapidly Degrading PEG-Based Thiol-Norbornene Hydrogels. ACS Macro Letters. 2021; 10:341-5.

Anderson S B, Lin C-C, Kuntzler D V, Anseth K S. The performance of human mesenchymal stem cells encapsulated in cell-degradable polymer-peptide hydrogels. Biomaterials. 2011;32:3564-74.

Lin C-C, Raza A, Shih H. PEG hydrogels formed by thiol-ene photo-click chemistry and their effect on the formation and recovery of insulin-secreting cell spheroids. Biomaterials. 2011;32:9685-95.

Shih H, Lin C-C. Cross-linking and degradation of step-growth hydrogels formed by thiol-ene photoclick chemistry. Biomacromolecules. 2012; 13:2003-12.

Jiang Z, Shaha R, McBride R, Jiang K, Tang M, Xu B, et al. Crosslinker length dictates step-growth hydrogel network formation dynamics and allows rapid on-chip photoencapsulation. Biofabrication. 2020;12:035006.

Mao A, Shin J-W, Utech S. Wang H, Uzun O, Li W, et al. Deterministic encapsulation of single cells in thin tunable microgels for niche modelling and therapeutic delivery. Nature materials. 2017;16:236-43.

Headen D M, Garcia J R, Garcia A J. Parallel droplet microfluidics for high throughput cell encapsulation and synthetic microgel generation. Microsystems & Nanoengineering. 2018;4:1-9.

Xia B. Jiang Z, Debroy D, Li D, Oakey J. Cytocompatible cell encapsulation via hydrogel photopolymerization in microfluidic emulsion droplets. Biomicrofluidics. 2017;11:044102.

Joshi J, Abnavi M D, Kothapalli C R. Synthesis and secretome release by human bone marrow mesenchymal stem cell spheroids within three-dimensional collagen hydrogels: Integrating experiments and modelling. Journal of tissue engineering and regenerative medicine. 2019;13:1923-37.

Ki C S, Lin T-Y. Kore M. Lin C-C. Thiol-ene hydrogels as desmoplasia-mimetic matrices for modeling pancreatic cancer cell growth, invasion, and drug resistance, Biomaterials. 2014:35:9668-77.

Lin T-Y, Ki C S, Lin C-C. Manipulating hepatocellular carcinoma cell fate in orthogonally cross-linked hydrogels. Biomaterials. 2014:35:6898-906.

Munoz Z, Shih H, Lin C-C. Gelatin hydrogels formed by orthogonal thiol-norbornene photochemistry for cell encapsulation. Biomaterials Science. 2014;2:1063-72.

Kuhn N Z, Toan R S. Regulation of stemness and stem cell niche of mesenchymal stem cells: implications in tumorigenesis and metastasis. Journal of cellular physiology. 2010;222:268-77.

Bian L. Guvendiren M, Mauck R L, Burdick J A. Hydrogels that mimic developmentally relevant matrix and N-cadherin interactions enhance MSC chondrogenesis. Proceedings of the National Academy of Sciences. 2013; 110:10117-22.

Kouroupis D, Correa D. Increased Mesenchymal Stem Cell functionalization in three-dimensional manufacturing settings for enhanced therapeutic applications. Frontiers in Bioengineering and Biotechnology, 2021;9.

Lee E J, Park S. J, Kang S K, Kim G-H, Kang H-J, Lee S-W, et al. Spherical bullet formation via E-cadherin promotes therapeutic potency of mesenchymal stem cells derived from human umbilical cord blood for myocardial infarction. Molecular Therapy. 2012;20:1424-33.

Hemmings B A, Restuccia D F. Pi3k-pkb/akt pathway. Cold Spring Harbor perspectives in biology. 2012;4:a011189.

Carracedo A, Pandolfi P. The PTEN-PI3K pathway: of feedbacks and cross-talks. Oncogene. 2008;27:5527-41.

Landi S, Moreno V, Gioia-Patricola L, Guino E, Navarro M, de Oca J, et al. Association of common polymorphisms in inflammatory genes interleukin (IL) 6, IL8, tumor necrosis factor α, NFKB1, and peroxisome proliferator-activated receptor y with colorectal cancer. Cancer research. 2003;63:3560-6.

Bernardo M, Fridman R. TIMP-2 (tissue inhibitor of metalloproteinase-2) regulates MMP-2 (matrix metalloproteinase-2) activity in the extracellular environment after pro-MMP-2 activation by MTI (membrane type 1)-MMP. Biochemical Journal. 2003:374:739-45.

Jiang Z. Xia B, McBride R. Oakey J. A microfluidic-based cell encapsulation platform to achieve high long-term cell viability in photopolymerized PEGNB hydrogel microspheres. Journal of Materials Chemistry B. 2017:5:173-80.

Zhang, K.; Wei, Z.; Xu, X.; Feng, Q.: Xu, J.; Bian, L. Efficient Catechol Functionalization of Biopolymeric Hydrogels for Effective Multiscale Bioadhesion. Mater. Sci. Eng. C 2019. 103. 109835.

Metters, A. T.; Bowman, C. N.; Anseth, K. S. A Statistical Kinetic Model for the Bulk Degradation of PLA-b-PEG-b-PLA Hydrogel Networks. J. Phys Chem. B 2000, 104 (30). 7043-7049.

Metters, A. T.; Anseth, K. S.; Bowman, C. N. A Statistical Kinetic Model for the Bulk Degradation of PLA-b-PEG-b-PLA Hydrogel Networks: Incorporating Network Non-Idealities. J. Phys. Chem. B 2001, 105 (34), 8069-8076.

While embodiments of the present disclosure have been described herein, it is to be understood by those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following clahos define the scope of the invention and that methods and structures within the scope of these claims and their equivalents be covered thereby.

1. A method for synthesizing a norbornene-functionalized polyethylene glycol macromer, comprising:
reacting a polyethylene glycol reactant, the polyethylene glycol reactant comprising at least one polyethylene glycol domain with a terminal hydroxyl group, with carbic anhydride in the presence of a nucleophilic catalyst to yield a norbornene-functionalized polyethylene glycol macromer having an ester linkage between a polyethylene glycol domain of the macromer and a norbornene domain of the macromer, wherein the norbornene domain comprises a carboxyl group.

2. The method of claim 1, wherein the polyethylene glycol reactant comprises a multi-arm polyethylene glycol molecule.

3. The method of claim 1, wherein the polyethylene glycol reactant comprises a polyethylene glycol molecule selected from the group consisting of a 2-arm polyethylene glycol molecule, a 3-arm polyethylene glycol molecule, a 4-arm polyethylene glycol molecule, a 6-arm polyethylene glycol molecule, an 8-arm polyethylene glycol molecule and combinations thereof.

4. The method of claim 1, wherein the nucleophilic catalyst comprises 4-dimethylaminopyridine.

5. The method of claim 1, wherein the norbornene-functionalized polyethylene glycol macromer comprises a structure as shown in Formula 1: [symbol] Formula 1
wherein
n=from about 10 to about 300, and
R₁ comprises a member selected from the group consisting of a second arm of PEG, optionally additional arms of PEG, a macromolecule, a PEGylated inorganic compound that includes a tail of PEG, and combinations thereof.

6. The method of claim 1, further comprising conjugating the carboxyl group of the norbornene domain with an amine-containing molecule to provide a modified macromer.

7. The method of claim 6, wherein the amine-containing molecule comprises a member selected from the group consisting of L-3,4-dihydroxyphenethylamine, tyramine, isopropylamine and combinations thereof.

8. The method of claim 6, wherein the modified macromer comprises at least one component having a structure as shown in Formula 2: 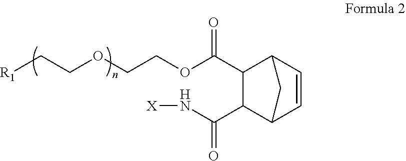
wherein
n=from about 10 to about 300,
X is an aromatic group or an aliphatic group, and
R₁ comprises a member selected from the group consisting of a second arm of PEG, optionally additional arms of PEG, a macromolecule, a PEGylated inorganic compound that includes a tail of PEG, and combinations thereof.

9. The method of claim 8, wherein X is selected from the group consisting of a functional group of Formula 3, a functional group of Formula 4, a functional group of Formula 5, a functional group of Formula 6, and a functional group of Formula 7:

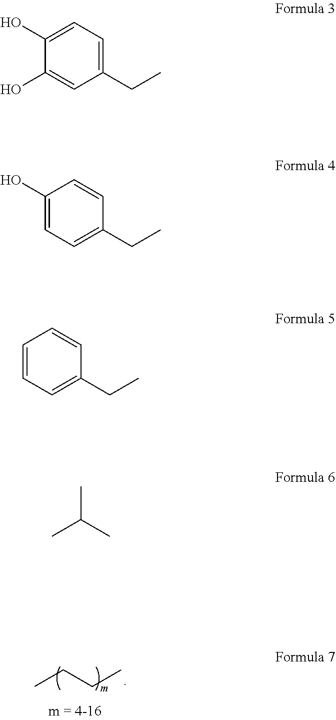

10. (canceled)

11. A macromer for producing a hydrogel, the macromer comprising a multi-arm polyethylene glycol core having at least two polyethylene glycol domains, each polyethylene glycol domain connected through an ester linkage to a norbornene moiety, wherein at least one norbornene moiety of the macromer comprises a carboxyl group.

12. The macromer of claim 11, wherein each of the at least two polyethylene glycol domains comprises from about 10 to about 300 —CH₂—CH₂—O— units.

13. The macromer of claim 11, wherein the multi-arm polyethylene glycol core has a number of polyethylene glycol domains selected from the group consisting of two, three, four, six and eight.

14. The macromer of claim 11, wherein the macromer comprises at least one component having a structure as shown in Formula 1: 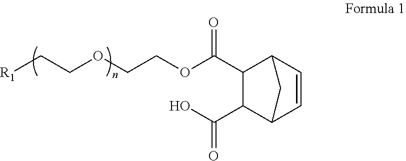
wherein
n=from about 10 to about 300, and
R₁ comprises a member selected from the group consisting of a second arm of PEG, optionally additional arms of PEG, a macromolecule, a PEGylated inorganic compound that includes a tail of PEG, and combinations thereof.

15. (canceled)

16. A macromer for producing a hydrogel, the macromer comprising a multi-arm polyethylene glycol core having at least two polyethylene glycol domains, each polyethylene glycol domain connected through an ester linkage to a norbornene moiety, wherein at least one norbornene moiety of the macromer is connected through a carboxamide linkage to a functional group.

17. The macromer of claim 16, wherein each of the at least two polyethylene glycol domains comprises from about 10 to about 300 —CH2—CH2—O— units.

18. The macromer of claim 16, wherein the multi-arm polyethylene glycol core has a number of polyethylene glycol domains selected from the group consisting of two, three, four, six and eight.

19. The macromer of claim 16, wherein the macromer comprises at least one component having a structure as shown in Formula 2: 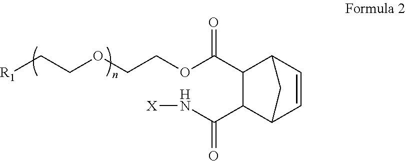
wherein
n=from about 10 to about 300,
X is an aromatic group or an aliphatic group, and
R₁ comprises a member selected from the group consisting of a second arm of PEG, optionally additional arms of PEG, a macromolecule, a PEGylated inorganic compound that includes a tail of PEG, and combinations thereof.

20. The macromer of claim 19, wherein X is selected from the group consisting of a functional group of Formula 3, a functional group of Formula 4, a functional group of Formula 5, a functional group of Formula 6, and a functional group of Formula 7:

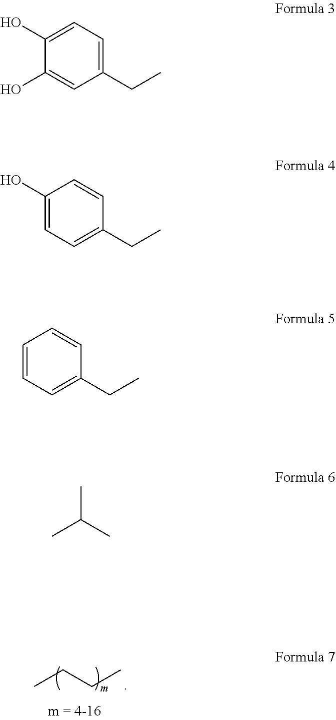

21. (canceled)

22. A method for making a hydrogel, comprising:
mixing a first quantity of macromers, the first quantity of macromers comprising a macromer according to claim 11, with a second quantity of dithiol molecules to provide a reaction mixture; and
applying ultraviolet radiation to the reaction mixture to initiate a photo-gelation reaction to yield a hydrogel.

23. (canceled)

24. The method of claim 22, wherein the dithiol molecule is selected from the group consisting of 1,4-dithiothreitol, 4-arm thiolated PEG, a peptide that includes more than one cysteine residue, a natural or thiolated, and thiolated collagens, and combinations thereof.

25. The method of claim 22, wherein the dithiol molecule comprises dithiothreitol.

26. The method of claim 22, wherein the hydrogel comprises an orthogonal thiol-norbornene hydrogel.

27. (canceled)

28. (canceled)

29. (canceled)

30. (canceled)

31. (canceled)

32. (canceled)

33. A method for forming a macroporous hydrogel, the macroporous hydrogel comprising at least one cell, the method comprising:
mixing a hydrogel-forming solution with a media containing the at least one cell, the hydrogel-forming solution comprising a crosslinkable material;
crosslinking at least a portion of the crosslinkable material of the hydrogel-forming solution, thereby forming a microgel comprising the at least one cell and a sacrificial material;
dispersing the microgel in a bulk hydrogel; and
removing the sacrificial material, thereby forming the macroporous hydrogel.

34. The method of claim 33, wherein the at least one cell comprises a plurality of cells, and at least a portion of the plurality of cells accumulate into a multicellular cluster in at least one pore of the macroporous hydrogel.

35. The method of claim 34, wherein the multicellular cluster is in a spheroidal structure.

36. (canceled)

37. (canceled)

38. The method of claim 33, wherein the at least one cell comprises at least one mesenchymal stem cell.

39. (canceled)

40. The method of any one of claim 33, wherein the crosslinkable material comprises at least one component having a structure as shown in Formula 2: 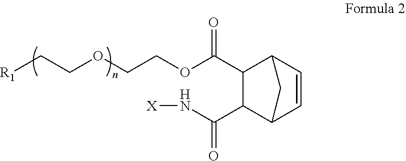
wherein
n=from about 10 to about 300,
X is an aromatic group or an aliphatic group, and
R₁ comprises a member selected from the group consisting of a second arm of PEG, optionally additional arms of PEG, a macromolecule, a PEGylated inorganic compound that includes a tail of PEG, and combinations thereof.

41. The method of claim 40, wherein X is selected from the group consisting of a functional group of Formula 3, a functional group of Formula 4, a functional group of Formula 5, a functional group of Formula 6, and a functional group of Formula 7:

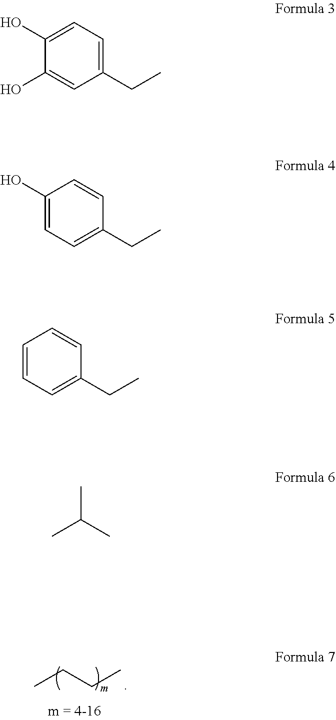

42. (canceled)


SEQUENCE LISTING (1 sequence)

SEQ ID NO: 1 - protein, 12 aa, Unknown
  Cleavage site at GI
KCGPQGIWGQCK